(19) United States
(12) Patent Application Publication
(10) Pub. No.: US 2023/0073558 A1
(43) Pub. Date: Mar. 9, 2023

(54) METHODS FOR PREDICTING AML OUTCOME

(71) Applicants: University of Florida Research Foundation, Incorporated, Gainesville, FL (US); St. Jude Children's Research Hospital, Inc., Memphis, TN (US); University of Tennessee Research Foundation, Knoxville, TN (US)

(72) Inventors: Jatinder Kaur Lamba, Gainesville, FL (US); Stanley Pounds, Memphis, TN (US); Abdelrahman H. Elsayed, Gainesville, FL (US); Xueyuan Cao, Memphis, TN (US)

(73) Assignees: University of Florida Research Foundation, Incorporated, Gainesville, FL (US); St. Jude Children's Research Hospital, Inc., Memphis, TN (US); University of Tennessee Research Foundation, Knoxville, TN (US)

(21) Appl. No.: 17/762,441

(22) PCT Filed: Sep. 22, 2020
(86) PCT No.: PCT/US2020/051961

§ 371 Date: Mar. 22, 2022

Related U.S. Application Data
(60) Provisional application No. 62/944,523, filed on Dec. 6, 2019.
(60) Provisional application No. 62/904,552, filed on Sep. 23, 2019.

(51) Int. Cl. C12Q 1/6886; G16B 25/10; A61P 35/02

(52) U.S. Cl. CPC C12Q 1/6886; G16B 25/10; A61P 35/02; C12Q 2600/158

(57) ABSTRACT

Aspects of the disclosure relate to compositions and methods for predicting prognosis and classifying risk of subjects having certain cancers, for example acute myeloid leukemia (AML). In some embodiments, methods described by the disclosure comprise a step of assessing the mRNA expression of certain leukemic stem cell (LSC)-enriched genes in a subject to produce a predictive score for pediatric AML. In some embodiments, methods described by the disclosure comprise a step of assessing the mRNA expression of certain genes of pharmacological relevance for standard chemotherapy consisting of Cytarabine (also known as Ara-C), daunorubicin and etoposide in a subject to produce a predictive score for pediatric AML.

### RELATED APPLICATIONS

This Application is a national stage filing under 35 U.S.C. § 371 of International Patent Application Serial No. PCT/US2020/051961, filed Sep. 22, 2020, which claims the benefit under 35 U.S.C. § 119(e) of the filing date of U.S. provisional application serial number 62/904,552, filed Sep. 23, 2019, entitled “PLSC6 SCORE PREDICTIVE OF HIGH RISK AML”, and U.S. provisional application Ser. No. 62/944,523, filed Dec. 6, 2019, entitled “METHODS FOR PREDICTING AML OUTCOME”, the entire contents of each of which are incorporated herein by reference in their entirety.

### FEDERALLY SPONSORED RESEARCH

This invention was made with government support under grant No. CA132946 awarded by the National Institutes of Health. The government has certain rights in the invention.

### BACKGROUND

Resistant and relapsed disease remain the most prevalent forms of failure in both pediatric and adult AML. Persistence of leukemic stem cells (LSCs) is a primary cause of AML relapse. LSCs are also associated with drug resistance.

For last forty years the standard induction treatment of AML patients involves ara-C (Cytarabine), daunorubicin and etoposide (ADE standard chemotherapy). However, development of drug resistance is one of the major causes treatment failure and relapse in pediatric AML patients. Thus differential levels of genes involved in the metabolism, activation, inactivation or disposition of ara-C, daunorubicin and etoposide in patients impacts therapeutic outcome as well as resistant and refractory disease resulting in dismal outcome.

### SUMMARY

Aspects of the disclosure relate to compositions and methods for predicting prognosis and classifying risk of subjects having certain cancers, for example acute myeloid leukemia (AML). In some embodiments, the AML is pediatric AML.

In some aspects, the disclosure provides a method for analyzing expression of RNA transcripts of genes in a human leukemia patient, the method comprising obtaining a biological sample from a subject who has or is suspected of having leukemia; extracting RNA from the biological sample; reverse transcribing RNA transcripts of a set of genes consisting of DNMT3B, GPR56, CD34, SOCS2, SPINK2, and FAM30A, and at least one reference gene, to produce a set of cDNAs; amplifying the cDNAs to produce amplification products; performing a gene expression assay to quantify the levels of the amplification products in the biological sample.

In some aspects, the disclosure provides a method for analyzing expression of RNA transcripts of genes in a human leukemia patient, the method comprising obtaining a biological sample from a subject who has or is suspected of having leukemia; extracting RNA from the biological sample; reverse transcribing RNA transcripts of a set of genes consisting of DCTD, CBR1, MPO, ABCC1, and TOP2A, and at least one reference gene, to produce a set of cDNAs; amplifying the cDNAs to produce amplification products; performing a gene expression assay to quantify the levels of the amplification products in the biological sample.

The disclosure is based, in part, on the use of regression modeling to assess the mRNA expression of certain leukemic stem cell (LSC)-enriched genes, and identification of a six-gene leukemic stem cell (LSC) score, termed “pLSC6”, that is predictive of pediatric AML prognosis and treatment outcomes. In some embodiments, pLSC6 scores described by the disclosure, have increased predictive power relative to previously utilized AML scoring systems, for example the LSC 17 scoring system.

Accordingly, in some aspects, method for obtaining a pLSC6 score result in a subject having leukemia, the method comprising measuring a level of an RNA transcript of each of a set of genes consisting of DNMT3B, GPR56, CD34, SOCS2, SPINK2, and FAM30A in a biological sample obtained from a subject having leukemia; normalizing the levels against a level of at least one reference RNA transcript in the biological sample to provide normalized levels each of the RNA transcripts; weighting each of the normalized levels of the set to produce a weighted set; calculating a pLSC6 score for the subject using the weighted set; and creating a report comprising the pLSC6 score.

In some embodiments, leukemia is acute myeloid leukemia (AML). In some embodiments, AML is pediatric AML. In some embodiments, a subject is less than 19 years of age.

In some embodiments, a pLSC6 score is useful for determining a prognosis of a cancer patient (e.g., a leukemia patient, such as an AML patient), for example as an indicator of event-free survival (EFS), overall survival (OS), or in assessing whether a patient is a candidate for transplantation therapy.

In some embodiments, the disclosure provides a method of predicting the likelihood of survival of a leukemia patient (e.g., a pediatric acute myeloid leukemia (AML) patient) without the recurrence of leukemia, the method comprising: measuring a level of an RNA transcript of each of a set of genes consisting of DNMT3B, GPR56, CD34, SOCS2, SPINK2, and FAM30A in a biological sample obtained from the subject; normalizing the levels against a level of at least one reference RNA transcript in the biological sample to provide normalized levels each of the RNA transcripts; weighting each of the normalized levels of the set to produce a weighted set; calculating a pLSC6 score for the subject using the weighted set; assigning a designation of “low-pLSC6” or a “high-pLSC6” score to the subject; and predicting the likelihood of survival without the recurrence of leukemia, wherein a “high-pLSC6” score is indicative of a reduced likelihood of survival without recurrence of leukemia relative to a “low-pLSC6” score.

In some aspects, the disclosure relates to the use of regression modeling to assess the mRNA expression of genes associated with pharmacokinetics (PK) and/or pharmacodynamics (PD) of certain anti-cancer therapeutics (e.g., cytarabine, daunorubicin, etoposide, or the combination of these drugs which is referred to as “ADE”), and identification of a five-gene score, termed “ADE-RS5” or “ADRS-5” (“AML Drug Resistance Score”), that is predictive of AML prognosis and treatment outcomes.

Accordingly, in some aspects, the disclosure provides a method for obtaining an ADE-RS5 score result in a subject having leukemia, the method comprising measuring a level of an RNA transcript of each of a set of genes consisting of DCTD, CBR1, MPO, ABCC1, and TOP2A in a biological sample obtained from a subject having leukemia; normalizing the levels against a level of at least one reference RNA transcript in the biological sample to provide normalized levels each of the RNA transcripts; weighting each of the normalized levels of the set to produce a weighted set; calculating an ADE-RS5 score (ADRS-5 score) for the subject using the weighted set; and creating a report comprising the ADE-RS5 score (ADRS-5 score).

In some aspects, the disclosure provides a method of predicting the likelihood of survival of an acute myeloid leukemia (AML) patient without the recurrence of leukemia, the method comprising measuring a level of an RNA transcript of each of a set of genes consisting of DCTD, CBR1, MPO, ABCC1, and TOP2A in a biological sample obtained from the subject; normalizing the levels against a level of at least one reference RNA transcript in the biological sample to provide normalized levels each of the RNA transcripts; weighting each of the normalized levels of the set to produce a weighted set; calculating an ADE-RS5 score (ADRS-5 score) for the subject using the weighted set; assigning a designation of “low-ADE-RS5” (low-ADRS-5″) or a “high-ADE-RS5” (“high-ADRS-5”) score to the subject; and predicting the likelihood of survival without the recurrence of leukemia, wherein a “high-ADE-RS5” (“high-ASRS-5”) score is indicative of a reduced likelihood of survival without recurrence of leukemia relative to a “low-ADE-RS5” (“low-ADRS-5”) score.

The disclosure is based, in part, on the recognition that integrating a pLSC6 score with an ADE-RS5 score results in improved treatment outcome prediction in AML patients. In some aspects, the disclosure provides a method for obtaining a pLSC6/ADE-RS5 score result in a subject having leukemia, the method comprising measuring a level of an RNA transcript of each of a set of genes consisting of DNMT3B, GPR56, CD34, SOCS2, SPINK2, and FAM30A in a biological sample obtained from a subject having leukemia; normalizing the levels against a level of at least one reference RNA transcript in the biological sample to provide normalized levels each of the RNA transcripts; weighting each of the normalized levels of the set to produce a first weighted set; calculating a pLSC6 score for the subject using the first weighted set; measuring a level of an RNA transcript of each of a set of genes consisting of DCTD, CBR1, MPO, ABCC1, and TOP2A in a biological sample obtained from a subject having leukemia; normalizing the levels against a level of at least one reference RNA transcript in the biological sample to provide normalized levels each of the RNA transcripts; weighting each of the normalized levels of the set to produce a second weighted set; calculating an ADE-RS5 score for the subject using the second weighted set; and creating a report comprising the pLSC6/ADE-RS5 score.

In some aspects, the disclosure provides a method of predicting the likelihood of survival of an acute myeloid leukemia (AML) patient without the recurrence of leukemia, the method comprising measuring a level of an RNA transcript of each of a set of genes consisting of DNMT3B, GPR56, CD34, SOCS2, SPINK2, and FAM30A in a biological sample obtained from the subject; normalizing the levels against a level of at least one reference RNA transcript in the biological sample to provide normalized levels each of the RNA transcripts; weighting each of the normalized levels of the set to produce a first weighted set; calculating a pLSC6 score for the subject using the first weighted set; assigning a designation of “low-pLSC6” or a “high-pLSC6” score to the subject; measuring a level of an RNA transcript of each of a set of genes consisting of DCTD, CBR1, MPO, ABCC1, and TOP2A in a biological sample obtained from the subject; normalizing the levels against a level of at least one reference RNA transcript in the biological sample to provide normalized levels each of the RNA transcripts; weighting each of the normalized levels of the set to produce a second weighted set; calculating an ADE-RS5 score for the subject using the second weighted set; assigning a designation of “low-ADE-RS5” or a “high-ADE-RS5” score to the subject; designating the patient as a “Low/Low:pLSC6/ADE-RS5”, “Low/High:pLSC6/ADE-RS5”, “High/Low:pLSC6/ADE-RS5”, or “High/High:pLSC6/ADE-RS5” patient; and predicting the likelihood of survival without the recurrence of leukemia, wherein a “High/High:pLSC6/ADE-RS5” patient is indicated to have a reduced likelihood of survival without recurrence of leukemia relative to a “Low/Low:pLSC6/ADE-RS5” patient.

In some embodiments, a biological sample is a blood sample, spinal fluid sample, or tissue sample. In some embodiments, a tissue sample comprises bone marrow cells and/or leukemic blast cells.

In some embodiments, an RNA transcript is an mRNA transcript.

In some embodiments, measuring comprises determining the RNA transcript level of each of the set of genes (e.g., DNMT3B, GPR56, CD34, SOCS2, SPINK2, and FAM30A, or DCTD, CBR1, MPO, ABCC1, and TOP2A) by a hybridization-based assay. In some embodiments, the hybridization-based assay comprises a microarray assay or quantitative RT-PCT.

In some embodiments, measuring comprises determining the RNA transcript level of each of the set of genes (e.g., DNMT3B, GPR56, CD34, SOCS2, SPINK2, and FAM30A, or DCTD, CBR1, MPO, ABCC1, and TOP2A) by a nucleic acid sequencing assay. In some embodiments, nucleic acid sequencing assay comprises nanopore sequencing, next-generation sequencing, high-throughput sequencing, or digital gene expression.

In some embodiments, a weighting step comprises fitting a COX-LASSO regression model to normalized levels of a set of genes (e.g., normalized levels of DNMT3B, GPR56, CD34, SOCS2, SPINK2, and FAM30A, or DCTD, CBR1, MPO, ABCC1, and TOP2A). In some embodiments, a weighted set comprises at least one of the following regression coefficient values: (DNMT3B×0.189), (GPR56×0.054), (CD34×0.0171), (SOCS2×0.141), (SPINK2×0.109), or (FAM30A×0.0516). In some embodiments, a weighted set comprises at least one of the following regression coefficient values: (0.128×DCTD), (0.099×TOP2A), (0.212 x ABCC1), (0.113×MPO), or (0.126×CBR1).

In some embodiments, a pLSC6 score is calculated using the following algorithm: pLSC6=(DNMT3B×0.189)+(GPR56 ×0.054)+(CD34×0.0171)+(SOCS2×0.141)+(SPINK2×0.109)+(FAM30A×0.0516). In some embodiments, an ADE-RS5 score is calculated using the following algorithm: ADE-R55=(0.128×DCTD)−(0.099×TOP2A)+(0.212×ABCC1)−(0.113×MPO)−(0.126×CBR1).

In some embodiments, a report designates a subject as a “low-pLSC6” or a “high-pLSC6” subject. In some embodiments, a report designates a subject as a “low-ADE-RS5” or a “high-ADE-RS5” subject. In some embodiments, a report designates a subject as a “Low/Low:pLSC6/ADE-RS5”, “Low/High:pLSC6/ADE-RS5”, “High/Low:pLSC6/ADE-RS5”, or “High/High:pLSC6/ADE-RS5” subject.

In some embodiments, a report designates a subject as a candidate for transplant therapy, for example hematopoietic stem cell transplantation (HSCT). In some embodiments, a subject is administered one or more drug selected from cytarabine, daunorubicin, and etoposide, or a combination thereof (e.g., ADE) after creation of the report.

In some aspects, the disclosure provides a system for assigning a pLSC6 score to a subject, comprising: (i) a detection apparatus, which is operably connected to, (ii) a computer containing executable instructions for measuring a level of an RNA transcript of each of a set of genes consisting of DNMT3B, GPR56, CD34, SOCS2, SPINK2, and FAM30A in a biological sample obtained from a subject having leukemia; normalizing the levels against a level of at least one reference RNA transcript in the biological sample to provide normalized levels each of the RNA transcripts; weighting each of the normalized levels of the set to produce a weighted set; calculating a pLSC6 score for the subject using the weighted set; and creating a report comprising the LSC6 score.

In some aspects, the disclosure provides a system for assigning an ADE-RS5 score to a subject, comprising: a detection apparatus, which is operably connected to a computer containing executable instructions for measuring a level of an RNA transcript of each of a set of genes consisting of DCTD, CBR1, MPO, ABCC1, and TOP2A in a biological sample obtained from a subject having leukemia; normalizing the levels against a level of at least one reference RNA transcript in the biological sample to provide normalized levels each of the RNA transcripts; weighting each of the normalized levels of the set to produce a weighted set; calculating an ADE-RS5 score for the subject using the weighted set; and creating a report comprising the ADE-RS5 score.

In some embodiments, a detection apparatus is a microplate reader, microarray scanner, or a sequencing machine.

### BRIEF DESCRIPTION OF DRAWINGS

FIG. 1 shows a flow chart describing one embodiment of a strategy to establish a pediatric-specific LSC score consisting of 6 genes, designated as “pLSC6”.

FIGS. 2A-2D show representative data indicating a Pediatric LSC6 (pLSC6) score based on six stem cell genes (e.g., DNMT3B, GPR56 and CD34, SOCS2, SPINK2 and FAM30A) is predictive of clinical outcomes in two independent cohorts of pediatric AML (AML02 and TARGET). Based on recursive portioning cutoff, patients were categorized according to their pLSC6 scores into two groups; low-pLSC6 (top line and values; around 60% of AML02, TARGET patients) and high pLSC6 (bottom line and values; around 40% of the patients). High-pLSC6 scores predict poor event free survival (EFS) (FIGS. 2A and 2C) and overall survival (OS) (FIGS. 2B and 2D) in AML02 and TARGET cohorts, respectively. Number of patients at risk during follow up period of 10 years is given and P-values are based on Cox-hazard models

FIGS. 3A-3F show representative data for Pediatric LSC6 (pLSC6) score and minimum residual disease (MRD) status after Induction 1 course of treatment. Patients found positive for residual leukemic cells after Induction 1 course of treatment (MRD-IND1>0.1%) had statistically significant higher distribution in the pLSC6 high score group as compared to the low-pLSC6 score group in AML02 (FIG. 3A) and TARGET (FIG. 3B) cohorts. P-value based on Chi-square test. Event free survival (FIGS. 3C and 3E) and overall survival (FIGS. 3D and 3F) probabilities by pLSC6 score and MRD status in AML02 467 (FIGS. 3C and 3D) and TARGET cohorts (FIGS. 3E and 3F) are shown. Top lines and values represent patients with low pLSC6 scores while bottom lines and values represent patients with high pLSC6. Solid lines represent MRD-negative patients and dashed lines MRD-positive patients.

FIGS. 4A-4B show representative data indicating pLSC6 score sub-classifies standard risk group patients by clinical outcome. Kaplan-Meier estimates of EFS by high-pLSC6 (bottom line and values) or low-pLSC6 (top line and values) score groups in AML02 (FIG. 4A) and TARGET (FIG. 4B) cohorts.

FIGS. 5A-5D show representative Forest plots of multivariable Cox-proportional hazard models showing pLSC6 score as an independent prognostic factor of EFS and OS in AML02 and Target cohorts. Hazard ratios and 95% confidence intervals Cis are listed next to each variable for EFS (FIGS. 5A and 5C) and OS (FIGS. 5B and 5D) in AML02 and TARGET-AML cohorts, respectively. Within each Forest plot, HR for each variable is depicted as a box and 95% CI are shown as horizontal lines. The vertical line crossing the value of 1 represents non-statistically significant effect, odds of less than one indicates better, whereas greater than 1 indicate worse effects.

FIGS. 6A-6D show Kaplan-Meier estimates of EFS (FIGS. 6A and 6C) and OS (FIGS. 6B and 6D) by pLSC6 score in standard and high-risk AML patients who did or did not receive hematopoietic stem cell transplantation (HSCT) in AML02 and TARGET cohorts, respectively. Solid lines: HSCT and dashed lines: Non-HSCT.

FIG. 7 shows data for frequency of gene representation investigated in 1000 boot-strapping models run with LASSO.

FIG. 8 shows a scatterplot demonstrating significant correlation of pLSC6-derived from U133A data (U133A_pLSC6) with RNAseq data (left: RNAseq_pLSC6, n=55 patients) and RT-PCR data (right: RTPCR_pLSC6, n=14).

FIG. 9 shows a Q-Q plot comparing probability distributions of the pLSC6 score computed using gene expression data of two different platforms; U133A array in AML02 cohort and RNA-Seq in TARGET cohorts.

FIGS. 10A-10D show distribution of pediatric LSC6 (pLSC6) score based on limited number of stem cell genes by risk group in AML02 cohort (FIG. 10A) and TARGET cohorts (FIG. 10B). Distribution of pLSC6 score groups by cytogenetic features in AML02 500 cohort (FIG. 10C) and TARGET cohorts (FIG. 10D) are also shown.

FIGS. 11A-11C show a comparison of LSC17 (FIG. 11A) score and pLSC6 score (FIG. 11B) in TARGET cohort for association with induction 1 MRD. FIG. 11C shows ROC curves demonstrating a comparison of pLSC6 and LSC17 vs. MRD1 in TARGET cohort.

FIGS. 12A-12H show representative data relating to ADE-RS5 scores. FIG. 12A shows patients in the high ADE-RS5 group had significantly worse EFS (HR=4.07(2.43-6.83), P<0.0001) and OS (HR=4.54(2.42-8.49), P<0.0001) compared to patients in the low ADE-RS5 group. FIG. 12B shows patients in the high ADE-RS5 group had a higher proportion of MRD1 positive patients (P=0.014). Representative data for validation in an independent COG cohort, where patients in the high score group demonstrated higher MRD1 positivity (P=0.0005; FIG. 12D) and inferior EFS (HR=1.33(1.06-1.65), P=0.012) and inferior OS (HR=1.38(1.065-1.8), P=0.015) as shown in FIG. 12C. Integrating both pLSC6 and ADE-RS5 scores together classifies patients into three groups: Group 1: Low_pLSC6 AND Low_ADE-RS55 (Low); Group 2: Low_pLSC6 AND High_ADE-RS5 OR High_pLSC6 AND Low_ADE-RS5 (Low/High); Group 3: High_pLSC6 AND High_ADE-RS55 (High). Patients in low/low pLSC6-ADE-RS5 group demonstrated better outcomes compared to the low-high and the high/high score groups (EFS in AML02 cohort; FIG. 12E and OS; FIG. 12F). pLSC6-ADE-RS5 response score groups, MRD1 status, risk groups, WBC at diagnosis, and age in AML02 cohort, high pLSC6-ADE-RS5 score group was found significantly associated with poor EFS (HR=6.0(2.71-13.2), P<0.00001; FIG. 12G) and was a significant predictor of poor OS (HR=8.3(2.9-24.0), P<0.00001; FIG. 12H).

### DETAILED DESCRIPTION OF INVENTION

Aspects of the disclosure relate to compositions and methods for analyzing expression of RNA transcripts of genes in a human leukemia patient. The disclosure is based, in part, on the use of regression modeling to assess the mRNA expression of certain leukemic stem cell (LSC)-enriched genes, and identification of a six-gene leukemic stem cell (LSC) score, termed “pLSC6”, that is predictive of pediatric AML prognosis and treatment outcomes. In some embodiments, pLSC6 scores described by the disclosure, have increased predictive power relative to previously utilized AML scoring systems, for example the LSC 17 scoring system.

In some aspects, the disclosure relates to the use of regression modeling to assess the mRNA expression of genes associated with pharmacokinetics (PK) and/or pharmacodynamics (PD) of certain anti-cancer therapeutics (e.g., Cytarabine (also known as ara-C), daunorubicin, etoposide, or the combination of these drugs which is referred to as “ADE”), and identification of a five-gene score, termed “ADE-RS5”(or in some instances ADE-RS), that is predictive of AML prognosis and treatment outcomes.

### Molecular Assays

Aspects of the disclosure relate to methods for analyzing expression of RNA transcripts of genes in a biological sample. In some embodiments, a biological sample is obtained from a subject.

As used herein, the term “subject” (or “patient”) refers to an animal having or suspected of having a disease, or an animal that is being tested for a disease. In some embodiments, the subject is selected from the group consisting of human, non-human primate, rodent (e.g., mouse or rat), canine, feline, or equine. In some embodiments, the subject is a human. In some embodiments a human subject is an adult (e.g., an individual over the age of 18). In some embodiments a subject is a child (e.g., a pediatric subject) that is less than 18 years of age. In some embodiments, a subject has previously been administered one or more anti-cancer agents, for example Cytarabine (or ara-C), daunorubicin, etoposide, or the combination of these drugs which is referred to as “ADE”.

In some embodiments, a subject (e.g., a human subject) has or is suspected of having a disease. A subject that “has or is suspected of having a disease” may exhibit one or more signs or symptoms of a particular disease (e.g., cancer), or may have been identified as having one or more genetic markers (e.g., genetic mutations, insertions, deletions, etc.) that increase the risk of the subject developing the disease (e.g., cancer). In some embodiments, the disease is a bacterial, viral, parasitic or autoimmune disease. In some embodiments, the disease is related to a mutation in the genome of the subject, for example cancer resulting from the mutation of a cancer suppressor gene. In some embodiments, the disease is related to a chromosomal abnormality, such as a chromosomal deletion, in the genome of the subject.

Generally, a biological sample can be blood, serum (e.g., plasma from which the clotting proteins have been removed), or cerebrospinal fluid (CSF). However, the skilled artisan will recognize other suitable biological samples, such as certain tissue (e.g., bone marrow, brain tissue, spinal tissue, etc.) and cells (e.g., leukocytes, stem cells, brain cells, neuronal cells, skin cells, etc.). In some embodiments, a biological sample is a blood sample or a tissue sample. In some embodiments, a blood sample is a sample of whole blood, a plasma sample, or a serum sample. In some embodiments, a tissue sample is a bone marrow tissue sample. In some embodiments, a blood sample is treated to remove white blood cells (e.g., leukocytes), such as the buffy coat of the sample. In some embodiments, a biological sample is obtained from a leukemia patient (e.g., a human leukemia patient). In some embodiments, a tissue sample comprises bone marrow cells and/or leukemic blast cells. In some embodiments, a tissue sample comprises bone marrow aspirate.

In some aspects, methods described by the disclosure include extraction and/or isolation of nucleic acids (e.g., DNA, RNA, miRNA, etc.) from a biological sample. Methods of extracting nucleic acids from a sample are known, for example as described in Ali et al. (2017) Biomed Res Int.:9306564. In some embodiments, RNA, such as mRNA is extracted from a biological sample. In some embodiments, total RNA is extracted from a biological sample using a commercially available RNA extraction kit, such as Qiagen RNeasy minicolumns, or Masterpure™ Complete DNA and RNA Purification Kit.

In some embodiments, methods described herein comprise a step of reverse transcribing RNA extracted from a biological sample to produce one or more cDNAs. The disclosure is based, in part, on reverse transcription and/or amplification of certain RNA transcripts relative to other RNA transcripts in the biological sample. In some embodiments, RNA transcripts of a set of genes consisting of DNMT3B, GPR56, CD34, SOCS2, SPINK2, and FAM30A, and at least one reference gene, are reverse transcribed to produce a set of cDNAs. In some embodiments, RNA transcripts of a set of genes consisting of DCTD, CBR1, MPO, ABCC1, and TOP2A, and at least one reference gene, are reverse transcribed to produce a set of cDNAs. In some embodiments, methods described herein comprise a step of amplifying the cDNAs to produce amplification products, also referred to as “amplicons”.

DNA Methyltransferase 3 Beta is an enzyme that is involved in DNA methylation. It is encoded by the DNMT3B gene in humans, for example as set forth in NCBI Reference Sequence Number NG_007290.1. Mutations in DNMT3B has previously been observed to be associated with leukemia, such as AML.

G protein-coupled receptor 56 (GPR56) is a member of the adhesion G protein-coupled receptor (GPCR) family. Adhesion GPCRs are characterized by an extended extracellular region often possessing N-terminal protein modules that is linked to a TM7 region via a domain known as the GPCR-Autoproteolysis INducing (GAIN) domain. It is encoded by the GPR56 gene in humans, for example as set forth in NCBI Reference Sequence Number NG_011643.1. High levels of GPR56 have been observed to associate with poor clinical outcomes in AML patients.

CD34 is a transmembrane phosphoglycoprotein protein encoded by the CD34 gene in humans, mice, rats and other species, which encodes an RNA transcript having the sequence set forth in NCBI Reference Sequence Number NM_001025109.2.

Suppressor of cytokine signaling 2 (SOCS2) is a protein that is a member of the STAT-induced STAT inhibitor (SSI), which is a cytokine-inducible negative regulator of cytokine signaling. It is encoded by SOCS2 gene in humans, which encodes an RNA transcript having the sequence set forth in NCBI Reference Sequence Number NM_001270467.2.

Serine protease inhibitor Kazal-type 2 (SPINK2) is a serine peptidase inhibitor, and in humans is encoded by the SPINK2 gene, which encodes an RNA transcript having the sequence set forth in NCBI Reference Sequence Number NM_001271718.2.

Family With Sequence Similarity 30 Member A (FAM30A) is a non-protein-coding RNA that is encoded by FAM30A, for example as set forth in NCBI Reference Sequence Number NG_001019.6.

Deoxycytidylate deaminase (DCTD) a deaminase, and in humans is encoded by the DCTD gene, for example as set forth in NCBI Reference Sequence Number NM_001012732.1. In some embodiments, DCTD is involved in ara-C (Cytarabine) inactivation.

Carbonyl reductase 1 (CBR1) is a carbonyl reductase that is encoded in humans by the CBR1 gene, which encodes an RNA transcript having the sequence set forth in NCBI Reference Sequence Number NM_001757 or NM_001286789. In some embodiments, CBR1 is involved in inactivation of daunorubicin (DNR).

Myeloperoxidase (MPO) is a peroxidase enzyme, and in humans is encoded by the MPO gene, which encodes an RNA transcript having the sequence set forth in NCBI Reference Sequence Number NM_000250. In some embodiments, myeloperoxidase is an etoposide activator.

Multidrug resistance-associated protein 1 (MRP1 or ABCC1) is an ATP-binding cassette transporter, which in humans is encoded by the ABCC1 gene, which encodes an RNA transcript having the sequence set forth in NCBI Reference Sequence Number NM_004996, NM_019862, NM_019898, NM_019899, or NM_019900. In some embodiments, ABCC1 is an efflux transporter of DNR and etoposide.

DNA topoisomerase 2-alpha (TOP2A) is an enzyme that alters topologic states of DNA, and in humans is encoded by the TOP2A gene, which encodes a RNA transcript having the sequence set forth in NCBI Reference Sequence Number NM_001067. In some embodiments, TOP2A is a target for DNR and etoposide.

As used herein, a “reference gene” is any gene which is constitutive genes that are required for the maintenance of basic cellular function, and are expressed in all cells of an organism under normal and patho-physiological conditions. Examples of reference genes include but are not limited to GAPDH (glyceraldehyde 3-phosphate dehydrogenase), SDHA (succinate dehydrogenase), HPRT1 (hypoxanthine phosphoribosyl transferase 1), HBS1L (HBS1-like protein), AHSP (alpha hemoglobin stabilizing protein), B2M (beta-2-microglobulin), etc. In some embodiments, at least 10, 20, 30, 40, 50, 60, 70, 80, 90, or 100 reference genes are reverse transcribed. In some embodiments, more than 100 reference genes are reverse transcribed.

In some embodiments, methods of the disclosure further comprise a step of performing a gene expression assay, for example to quantify the levels of certain amplification products or RNA transcripts in the biological sample. A “gene expression assay” refers to a molecular, biological, or chemical assay which quantifies the relative expression level of a particular gene relative to other genes. In some embodiments, a gene expression assay quantifies the relative expression level of a particular set of genes relative to either 1) other genes or 2) each other gene in the set. Expression levels of genes may be determined by quantifying a level of DNA, RNA (e.g., total RNA, mRNA, miRNA, etc.), or proteins translated as a result of expression of the gene or set of genes.

In some embodiments, a gene expression profile of a sample is determined by quantitative reverse transcription polymerase chain reaction (qRT-PCR). Briefly, mRNA is isolated from a biological sample is reverse transcribed (for example by Moloney murine leukemia virus (MMLV) reverse transcriptase) and subsequently amplified using gene specific primers and a thermostable DNA-dependent DNA polymerase, such as Taq DNA polymerase. A number of commercial qRT-PCR assay kits are commercially available, for example SYBR Green, Taqman, and Molecular Beacons. qRT-PCR protocols are described, for example in Bustin (2002) Journal of Molecular Endocrinology, 29, 23-39.

In some embodiments, a gene expression profile of a sample is determined by a microarray assay. Microarray assays are known, for example as described in Bumgartner (2013) Curr Protoc Mol Biol. 2013 Jan; 0 22: Unit-22.1. Examples of commercially available microarray assays include Affymetrix GeneChip, Illumina BeadArray, Agilent microarrays, etc. Generally, a microarray assay comprises the steps of detecting the presence or absence of an interaction between a sample (e.g., a nucleic acid such as RNA or cDNA present in a sample) and a material at each location on a substrate. Various methods of detecting an interaction are recognized in the art. For example, interaction between the sample and the material can be detected by measuring binding activity between the sample and the material.

As used herein, the term “binding activity” refers to the chemical linkage formed between two molecules. For example, a protein ligand may become covalently bound to its cognate receptor via the chemical interaction between the amino acid residues of the ligand and the receptor. In the context of nucleic acid interactions, binding activity includes the hybridization of complementary nucleic acids. As used herein, the term “hybridization” is accorded its general meaning in the art and refers to the pairing of substantially complementary nucleotide sequences (for example, pairing of oligonucleotides and strands of nucleic acid) to form a duplex or heteroduplex through formation of hydrogen bonds between complementary base pairs in accordance with Watson-Crick base pairing. Hybridization is a specific, i.e., non-random, interaction between two complementary polynucleotides.

In some embodiments, the gene expression profile of a sample is determined by nucleic acid sequencing (e.g., DNA sequencing, RNA sequencing, etc.). Examples of sequencing methods used for gene expression profiling include but are not limited to single-molecule real-time sequencing (SMRT), ion semiconductor (Ion Torrent) sequencing, pyrosequencing, sequencing by synthesis (e.g., Illumina sequencing), sequencing by ligation (SOLiD), and chain termination sequencing (Sanger sequencing), nanopore sequencing (e.g., Oxford Nanopore sequencing), and massively parallel sequencing (MPSS). Sequencing methods generally utilize gene specific probes (e.g., oligonucleotides, primers, adaptors, etc.) for nucleic acid amplification. In some embodiments, gene-specific probes selectively hybridize to a gene selected from DNMT3B, GPR56, CD34, SOCS2, SPINK2, and FAM30A. In some embodiments, gene-specific probes selectively hybridize to a gene selected from DCTD, CBR1, MPO, ABCC1, and TOP2A.

Aspects of the disclosure relate to kits comprising: a first oligonucleotide that hybridizes to a portion of a DNMT3B nucleic acid; a second oligonucleotide that hybridizes to a portion of a GPR56 nucleic acid; a third oligonucleotide that hybridizes to a portion of a CD34 nucleic acid; a fourth oligonucleotide that hybridizes to a portion of a SOCS2 nucleic acid; a fifth oligonucleotide that hybridizes to a portion of a SPINK2 nucleic acid; and a sixth oligonucleotide that hybridizes to a portion of a FAM30A nucleic acid.

Aspects of the disclosure relate to kits comprising: a first oligonucleotide that hybridizes to a portion of a DCTD nucleic acid; a second oligonucleotide that hybridizes to a portion of a CBR1 nucleic acid; a third oligonucleotide that hybridizes to a portion of a MPO nucleic acid; a fourth oligonucleotide that hybridizes to a portion of a ABCC1 nucleic acid; and a fifth oligonucleotide that hybridizes to a portion of a TOP2A nucleic acid.

In some embodiments, each of the first, second, third, fourth, fifth, and sixth oligonucleotides are housed in the same container. In some embodiments, each of the first, second, third, fourth, fifth, and, optionally, sixth oligonucleotides are housed in different containers.

In some embodiments, one or more oligonucleotides of a kit comprises a detectable label or a sequencing adaptor molecule. In some embodiments, a detectable label is a fluorescent moiety, luminescent moiety, or an enzyme. In some embodiments, a kit further comprises one or more containers housing one or more buffer solutions.

### Scoring

Aspects of the disclosure relate to methods for determining a prognosis of a cancer patient and/or determining whether a patient is an appropriate candidate for transplantation (e.g., hematopoietic stem cell transplantation, HSCT). Without wishing to be bound to any particular theory, methods described by the disclosure have a higher predictive power than previously described leukemia risk stratification algorithms, for example the LSC17 stemness score described by Ng et al. (2016) Nature, 540(7633):433-437. In some embodiments, methods of calculating a pLSC6 score comprise measuring a level of a nucleic acid (e.g., a gene, a cDNA, an RNA transcript, etc.) of each of a set of genes consisting of DNMT3B, GPR56, CD34, SOCS2, SPINK2, and FAM30A in a biological sample obtained from a subject having leukemia. In some embodiments, methods of calculating an ADE-RS5 score comprise measuring a level of a nucleic acid (e.g., a gene, a cDNA, an RNA transcript, etc.) of each of a set of genes consisting of DCTD, CBR1, MPO, ABCC1, and TOP2A in a biological sample obtained from a subject having leukemia. In some embodiments, the RNA levels of each of the set of genes is determined using a microarray assay, a nucleic acid sequencing assay, or a hybridization-based assay (e.g., a RT-PCR assay, a qPCR assay, a qRT-PCR assay, etc.).

Expression levels of nucleic acids may be normalized in order to minimize the effect of sample-to-sample variation or amplification errors. “Normalizing” refers to the transformation of raw expression data (e.g., data relating to detection of nucleic acid levels) to fit within a specified range. Methods for normalization of gene expression data depend on the modality used to collect the raw expression data. In some embodiments, normalization of qPCR data comprises the delta-delta-Ct (ΔΔCt), qBase, or methods described by Pfaffl (2001) Nucleic Acids Research 29(9):e45. In some embodiments, normalization of RNA-sequencing (RNA-seq) data comprises library size normalization methods (e.g., UQ, TMM, and RLE), or across-sample normalization methods (e.g., SVA, RUV, and PCA). In some embodiments, normalization of microarray gene expression data comprises RMA normalization, Mas 5.0 normalization, Quantile normalization, or invariant set normalization. In some embodiments, expression levels of a set of genes (e.g., DNMT3B, GPR56, CD34, SOCS2, SPINK2, and FAM30A, or DCTD, CBR1, MPO, ABCC1, and TOP2A) are normalized to produce a “normalized set of genes”. In some embodiments, each of the gene expression levels in the normalized set of genes has been normalized with respect to one or more reference genes (e.g., housekeeping genes), for example 1, 5, 10, 50, or 100 reference genes.

“Weighting” refers to an assignment of a value corresponding to a higher or lower importance to a member of a group. In some embodiments, a higher numerical value indicates an increased weight (e.g., higher significance) of a group member. In some embodiments, a higher numerical value indicates a decreased weight (e.g., lower significance) of a group member. In some embodiments, weighting a set of normalized genes comprises applying a regression model to the normalized set of genes. Examples of regression models include but are not limited to linear regression, non-linear regression, Bayesian regression, least absolute deviations, nonparametric regression, etc. In some embodiments, a regression model is a Cox regression model, for example as described by Cox (1972) J R Statist Soc B 34: 187-220. In some embodiments, a regression model comprises a “lasso” method, for example as described by Tibshirani (1997) Statistics in Medicine, 16:385-395.

Applying a weighting method (e.g., a linear regression model, such as a Cox-lasso model) to a normalized set of gene expression data (e.g., normalized levels of DNMT3B, GPR56, CD34, SOCS2, SPINK2, and FAM30A, or DCTD, CBR1, MPO, ABCC1, and TOP2A) produces a “weighted set” of gene expression data. A weighted set of gene expression values comprises a gene expression value multiplied by a regression coefficient (e.g., a value derived from applying the weighting to the set of normalized genes).

In some embodiments, a regression coefficient ranges in value from about 0.01 to about 0.23 (e.g., any value between 0.01 and 0.23, inclusive). In some embodiments, a DNMT3B gene expression value is multiplied by a regression coefficient ranging from about 0.151 to about 0.23. In some embodiments, a GPR56 gene expression value is multiplied by a regression coefficient ranging from about 0.043 to about 0.065. In some embodiments, a CD34 gene expression value is multiplied by a regression coefficient ranging from about 0.0136 to about 0.021. In some embodiments, a SOCS2 gene expression value is multiplied by a regression coefficient ranging from about 0.113 to about 0.169. In some embodiments, a SPINK2 gene expression value is multiplied by a regression coefficient ranging from about 0.087 to about 0.131. In some embodiments, a FAM30A gene expression value is multiplied by a regression coefficient ranging from about 0.041 to about 0.062. In some embodiments, a weighted set comprises at least one of the following regression coefficient values: (DNMT3B×0.189), (GPR56 ×0.054), (CD34×0.0171), (SOCS2×0.141), (SPINK2×0.109), or (FAM30A×0.0516).

In some embodiments, a pLSC6 score is calculated using the following algorithm: pLSC6=(DNMT3B×0.189)+(GPR56 ×0.054)+(CD34×0.0171)+(SOCS2×0.141)+(SPINK2×0.109)+(FAM30A×0.0516), where each gene name represents a normalized expression level value.

In some embodiments, a DCTD gene expression value is multiplied by a regression coefficient ranging from about 0.010 to about 0.154. In some embodiments, a CBR1 gene expression value is multiplied by a regression coefficient ranging from about 0.101 to about 0.151. In some embodiments, a MPO gene expression value is multiplied by a regression coefficient ranging from about 0.090 to about 0.136. In some embodiments, a ABCC1 gene expression value is multiplied by a regression coefficient ranging from about 0.170 to about 0.254. In some embodiments, a TOP2A gene expression value is multiplied by a regression coefficient ranging from about 0.079 to about 0.119. In some embodiments, a weighted set comprises at least one of the following regression coefficient values: (0.128×DCTD), (0.0993×TOP2A), (0.212×ABCC1), (0.113×MPO), or (0.126×CBR1).

In some embodiments, an ADE-RS5 score is calculated using the following algorithm:

ADE-RS5=(0.128×DCTD)−(0.0993×TOP2A)+(0.212×ABCC1)−(0.113×MPO)−(0.126×CBR1), where each gene name represents a normalized expression level value. A skilled person recognizes that the algorithm for calculating an ADE-RS5 score may alternatively be expressed as: (0.128×DCTD)+(−0.099×TOP2A)+(0.212×ABCC1)+(−0.113×MPO)+(−0.126×CBR1).

In some embodiments, a pLSC6 score , ADE-RS5 score, or pLSC6-ADE-RS5 score is useful for determining a prognosis of a cancer patient (e.g., a leukemia patient, such as an AML patient), for example as an indicator of event-free survival (EFS), overall survival (OS), or in assessing whether a patient is a candidate for transplantation therapy. In some embodiments, a pLSC6 score calculated (based on an RNAseq analysis) between about 0 and about 2 is assigned a designation of a “low pLSC6 score”. In some embodiments, a pLSC6 score calculated (based on a U133A array) between about 0 and about 4 is assigned a designation of a “low pLSC6 score”. In some embodiments, a pLSC6 score calculated (based on an RNAseq analysis) between about 1.5 and about 3 is assigned a designation of a “high pLSC6 score”. In some embodiments, a pLSC6 score calculated (based on a U133A array) between about 3 and about 5 is assigned a designation of a “high pLSC6 score”.

In some embodiments a pLSC6 score below 1.58 (e.g., as measured by a RNAseq platform) or 3.41 (e.g., as measured by a U133A array) is assigned a designation of a “low-pLSC6” score. In some embodiments, a pLSC6 score above 1.59 (e.g., as measured by a RNAseq platform) or 3.41 (e.g., as measured by a U133A array) is assigned a designation of a “high-pLSC6” score.

In some embodiments, an ADE-RS5 (ADRS-5) score calculated (e.g., based on an Illumina paired end high depth read RNAseq analysis) between about −0.964 and about 0.045 is assigned a designation of a “low ADE-RS5 score”. In some embodiments, an ADE-RS5 score calculated (e.g., based on an low depth RNAseq analysis) below 0.147 is assigned a designation of a “low ADE-RS5 score”. In some embodiments, an ADE-RS5 score calculated (e.g., based on RNAseq analysis after z-score transformation) below 0.178 is assigned a designation of a “low ADE-RS5 score”. In some embodiments, an ADE-RS5 score calculated (e.g., based on a U133A array) between about -0.504 and about 0.293 is assigned a designation of a “low ADE-RS5 score”. In some embodiments, an ADE-RS5 score calculated (e.g., based on an paired end high depth RNAseq analysis) between about 0.047 and about 1.43 is assigned a designation of a “high ADE-RS5 score”. In some embodiments, an ADE-RS5 score calculated (e.g., based on an RNAseq analysis at low depth) above 0.147 is assigned a designation of a “high ADE-RS5 score”. In some embodiments, an ADE-RS5 score calculated (e.g., based on RNAseq analysis after z-score transformation) above 0.179 is assigned a designation of a “high ADE-RS5 score”. In some embodiments, an ADE-RS5 score calculated (e.g., based on a U133A array) between about 0.298 and about 1.42 is assigned a designation of a “high ADE-RS5 score”.

Aspects of the disclosure relate to the recognition that pLSC6 and ADE-RS5 scores may be integrated in order to improve predictive value. Thus, in some embodiments, a subject is designated as a “Low/Low:pLSC6/ADE-RS5”, “Low/High:pLSC6/ADE-RS5”, “High/Low:pLSC6/ADE-RS5”, or “High/High:pLSC6/ADE-RS5” subject. In some embodiments (e.g., using U133A), a “Low/Low:pLSC6/ADE-RS5” subject has a pLSC6 score below about 3.41 (e.g., ranging from about 2.43-3.41), and an ADE-RS5 score below about 0.293 (e.g., ranging from about −0.504 and 0.293). In some embodiments (e.g., using U133A), a “Low/High:pLSC6/ADE-RS5” subject has a pLSC6 score below about 3.41 (e.g., ranging from about 2.43-3.41), and an ADE-RS5 score about above 0.298 (e.g., ranging from about 0.298-1.42). In some embodiments (e.g., using U133A), a “Low/High:pLSC6/ADE-RS5” subject has a pLSC6 score above about 3.45 (e.g., ranging from about 3.45-4.4), and an ADE-RS5 score ranging below about 0.293 (e.g., ranging about −0.504 to 0.293). In some embodiments (e.g., using U133A), a “High/High:pLSC6/ADE-RS5” subject has a pLSC6 score above about 3.45 (e.g., ranging from about 3.45-4.4), and an ADE-RS5 score above about 0.298 (e.g., ranging from about 0.298-1.42). Table 1 below summarizes the score ranges.

TABLE 1

| Platform | Low-pLSC6 | High-pLSC6 | Low-ADE-RS | High-ADE-RS5 |
|---|---|---|---|---|
| U133A | (2.43 to 3.41) | (3.45 to 4.4) | (−0.504 to 0.293) | (0.298 to 1.42) |
| RNASeq (illumina | (0.154 to 1.58) | (1.59 to 3.08) | (−0.964 to 0.045) | (0.047 to 1.43) |
| paired end -High | | | | |
| depth) | | | | |
| RNASeq (illumina low | (0.089 to 1.746) | (1.751 to 3.71) | (−1.35 to 0.1471) | (0.1473 to 1.55) |
| depth depth) | | | | |
| RNASeq (z- | (−1.96 to 0.183) | (0.185 to 2.74) | (−2.76 to 0.178) | (0.179 to 3.33) |
| transformation for | | | | |
| combining the | | | | |
| different read depths) | | | | |

In some embodiments, the method comprises a step of predicting the likelihood of survival of a subject without the recurrence of leukemia. In some embodiments, a high-pLSC6 score, high ADE-RS5 score, or High/High pLSC6/ADE-RS5 score is indicative of a reduced likelihood of survival without recurrence of leukemia relative to a low-pLSC6 score, low ADE-RS5 score, or Low/Low pLSC6/ADE-RS5 score. A subject having a reduced likelihood of survival (e.g., a subject having a high pLSC6 score, high ADE-RS5 score, or High/High pLSC6/ADE-RS5 score) may have about a 1%, 5%, 10%, 20%, 50%, 75%, 90%, 95%, or 99% increased probability of recurrence of cancer relative to a subject that does not have a reduced likelihood of survival (e.g., a subject having a low pLSC6 score, low ADE-RS5 score, or Low/Low pLSC6/ADE-RS5 score).

The term “prognosis” refers to the prediction of the likelihood of death attributable to cancer or progression of cancer, including recurrence, metastatic spread, and drug resistance of a neoplastic disease, such as leukemia. In some embodiments, leukemia is acute myeloid leukemia (AML), for example adult AML or pediatric AML.

As used herein, “event free survival” and “EFS” refers to the length of time after primary treatment for a cancer ends (e.g., after primary treatment of a leukemia ends) that the patient remains free of certain complications or events that the treatment was intended to prevent or delay, for example return of the cancer or onset of certain symptoms (e.g., bone pain from cancer that has spread to a bone). In some embodiments, a subject having a reduced likelihood of event free survival (e.g., a subject having a high pLSC6 score, high ADE-RS5 score, or High/High pLSC6/ADE-RS5 score) may have about a 1%, 5%, 10%, 20%, 50%, 75%, 90%, 95%, or 99% increased probability of recurrence of cancer relative to a subject that does not have a reduced likelihood of event free survival (e.g., a subject having a low pLSC6 score, low ADE-RS5 score, or Low/Low pLSC6/ADE-RS5 score).

As used here, “overall survival” and “OS” refers to the length of time from either the date of diagnosis or the start of treatment for a disease, such as cancer, that patients diagnosed with the disease are still alive. A subject having a reduced likelihood of overall survival (e.g., a subject having a “high pLSC6” score, high ADE-RS5 score, or High/High pLSC6/ADE-RS5 score) may have about a 1%, 5%, 10%, 20%, 50%, 75%, 90%, 95%, or 99% increased probability of dying prior to a subject that does not have a reduced likelihood of overall survival (e.g., a subject having a “low pLSC6” score, low ADE-RS5 score, or Low/Low pLSC6ADE-RS5 score).

As used herein, “minimum residual disease” and “MRD” refer to small numbers of leukemic cells that remain in a subject during treatment, or after treatment, when the patient is in remission (e.g., has no symptoms or signs of disease). MRD testing is typically used to determine if a treatment has eradicated the cancerous cells (e.g., cancerous bone marrow cells) or whether small populations of cancerous cells remain. In some embodiments, MRD testing is used to detect recurrence of the leukemia in a subject. Generally detection of more than 1 cancerous cell out of 1,000 cells in a sample indicates a “high” MRD, and a poor patient prognosis. The disclosure is based, in part, on the recognition that methods of detecting leukemic stem cells described herein display improved sensitivity relative to currently used MRD testing methods. In some embodiments, a pLSC6 assay is about 1%, 5%, 10%, 20%, 50%, 75%, 90%, 95%, or 99% more sensitive in identifying subjects likely to have recurrent leukemia relative to a MRD assay.

Therapeutic Methods Aspects of the disclosure relate to methods for diagnosing a subject as having (or being at risk of developing) certain cancers, such as leukemia. In some embodiments, the disclosure provides a method of diagnosing a subject has having leukemia, the method comprising detecting in a biological sample obtained from a subject that has been administered a cancer therapy a level of an RNA transcript of each of a set of genes consisting of DNMT3B, GPR56, CD34, SOCS2, SPINK2, and FAM30A in a biological sample obtained from the subject. In some embodiments, the level of each RNA transcript in the set of genes is normalized (e.g., against a level of one or more reference genes). In some embodiments, the normalized levels are weighted according to an algorithm described by the disclosure. In some embodiments, the weighted, normalized levels produce a pLSC6 score, which is indicative of the subject having cancer (e.g., leukemia, such as AML). In some embodiments, the method further comprises administering one or more anti-cancer therapeutics and/or radiation treatment, to the subject based upon the assignment of the pLSC6 score.

The disclosure relates, in some aspects, to methods of monitoring a therapeutic treatment course for a cancer, for example leukemia (e.g., AML), in a subject. In some embodiments, a subject has been administered one or more chemotherapeutics and/or has undergone one or more radiation treatments prior to providing the biological sample.

In some aspects, the disclosure provides methods of monitoring a cancer (e.g., leukemia) treatment comprising detecting in a biological sample obtained from a subject that has been administered a cancer therapy a level of an RNA transcript of each of a set of genes consisting of DNMT3B, GPR56, CD34, SOCS2, SPINK2, and FAM30A in a biological sample obtained from a subject having leukemia; normalizing the levels against a level of at least one reference RNA transcript in the biological sample to provide normalized levels each of the RNA transcripts; weighting each of the normalized levels of the set to produce a weighted set; calculating a pLSC6 score for the subject using the weighted set, and if the subject has a low pLSC6 score designating the subject as a candidate for hematopoietic stem cell transplant (HSCT).

In some embodiments, the method comprises administering one or more additional cancer therapeutics to the subject if the subject has a high pLSC6 score. Examples of cancer therapeutics include but are not limited to Arsenic Trioxide, Cerubidine (Daunorubicin Hydrochloride), Cyclophosphamide, Cytarabine, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS, Idarubicin, Idhifa , Ivosidenib, Midostaurin, Mitoxantrone Hydrochloride, Rydapt (Midostaurin), Thioguanine, Tibsovo (Ivosidenib), Venetoclax, and Vincristine Sulfate.

Aspects of the disclosure relate to methods of predicting outcome of treatment of a subject having AML with ADE therapy (e.g., Cytarabine, Daunorubicin, and etoposide). In some embodiments, a subject having a low ADE-RS5 score has a higher probability of a successful treatment outcome (e.g., reduction of tumor size, cancer cell death, reduction of symptoms, increased overall survival, etc.) relative to a subject having a high ADE-RS5 score. In some embodiments, a subject having a low ADE-RS5 score is administered one or more (e.g., 2, 3, 4, 5, or more) ADE doses after it is determined that the subject has a low ADE-RS5 score.

### Kits

Aspects of the disclosure relate to kits for detecting expression level of one or more transcripts in a biological sample.

In some aspects, the disclosure provides a kit comprising: a first oligonucleotide that hybridizes to a portion of a DNMT3B DNA transcript; a second oligonucleotide that hybridizes to a portion of a GPR56 DNA transcript; a third oligonucleotide that hybridizes to a portion of a CD34 DNA transcript; a fourth oligonucleotide that hybridizes to a portion of a SOCS2 DNA transcript; a fifth oligonucleotide that hybridizes to a portion of a SPINK2 DNA transcript; and a sixth oligonucleotide that hybridizes to a portion of a FAM30A DNA transcript.

In some embodiments, an oligonucleotide primer that hybridizes to DNMT3B comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth in NCBI Reference Sequence Number NG_007290.1.

In some embodiments, an oligonucleotide primer that hybridizes to GPR56 comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth in NCBI Reference Sequence Number NG_011643.1. High levels of GPR56 have been observed to associate with poor clinical outcomes in AML patients.

In some embodiments, an oligonucleotide primer that hybridizes to CD34 comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence encoding an RNA transcript having the sequence set forth in NCBI Reference Sequence Number NM_001025109.2.

In some embodiments, an oligonucleotide primer that hybridizes to SOCS2 comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence encoding an RNA transcript having the sequence set forth in NCBI Reference Sequence Number NM_001270467.2.

In some embodiments, an oligonucleotide primer that hybridizes to SPINK2 comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence encoding an RNA transcript having the sequence set forth in NCBI Reference Sequence Number NM_001271718.2.

In some embodiments, an oligonucleotide primer that hybridizes to FAM30A comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth set forth in NCBI Reference Sequence Number NG_001019.6.

In some aspects, the disclosure provides kits comprising: a first oligonucleotide that hybridizes to a portion of a DCTD nucleic acid; a second oligonucleotide that hybridizes to a portion of a CBR1 nucleic acid; a third oligonucleotide that hybridizes to a portion of a MPO nucleic acid; a fourth oligonucleotide that hybridizes to a portion of a ABCC1 nucleic acid; and a fifth oligonucleotide that hybridizes to a portion of a TOP2A nucleic acid.

In some embodiments, an oligonucleotide primer that hybridizes to DCTD comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth in NCBI Reference Sequence Number NM_001012732.1.

In some embodiments, an oligonucleotide primer that hybridizes to CBR1 comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth in NCBI Reference Sequence Number NM_001757 or NM_001286789.

In some embodiments, an oligonucleotide primer that hybridizes to MPO comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth in NCBI Reference Sequence Number NM_000250.

In some embodiments, an oligonucleotide primer that hybridizes to ABCC1 comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth in NCBI Reference Sequence Number NM_004996, NM_019862, NM_019898, NM_019899, or NM_019900.

In some embodiments, an oligonucleotide primer that hybridizes to TOP2A comprises a nucleic acid sequence that is complementary to between about 3 and about 30 (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) nucleotides of a sequence set forth in NCBI Reference Sequence Number NM_001067.

Design of oligonucleotide primers is known, for example as described by Jung et al. (2013) RNA 19: 1864-1873, or using Primer Express™ software (Applied Biosystems™).

In some embodiments, each of the first, second, third, fourth, fifth, and sixth oligonucleotides are housed in the same container. In some embodiments, each of the first, second, third, fourth, fifth, and sixth oligonucleotides are housed in different containers.

In some embodiments, one or more oligonucleotides of a kit comprises a detectable label or a sequencing adaptor molecule. In some embodiments, a detectable label is a fluorescent moiety, luminescent moiety, or an enzyme. In some embodiments, a kit further comprises one or more containers housing one or more buffer solutions.

### Computer Systems

Techniques as described herein may yield more accurate diagnosis and treatment recommendations for specific subjects. Such techniques involve collecting and processing data on a sufficient number of genes (e.g., DNMT3B, GPR56, CD34, SOCS2, SPINK2, and FAM30A, or DCTD, CBR1, MPO, ABCC1, and TOP2A) to produce data sets including adequate information to calculate a pLSC6 score using an algorithm described herein. The collection and/or processing of such data may be controlled by execution of a computing device.

The invention is operational with numerous other general purpose or special purpose computing system environments or configurations. Examples of well-known computing systems, environments, and/or configurations that may be suitable for use with the invention include, but are not limited to, personal computers, server computers, smartphones, tablets, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. Some embodiments may also be practiced in distributed computing environments where tasks are performed by remote processing devices that are linked through a communications network. These distributed systems may be what are known as enterprise computing systems or, in some embodiments, may be “cloud” computing systems. In a distributed computing environment, program modules may be located in both local and/or remote computer storage media including memory storage devices.

In some embodiments, a system comprises a detection apparatus. In some embodiments, a detection apparatus is a microplate reader (e.g., fluorescence microplate reader, UV microplate reader, photometer microplate reader, etc.), or a sequencing machine (e.g., a nanopore sequencing machine, a Next-generation sequencing machine, a RNA-seq machine, etc.). In some embodiments, the detection apparatus is electronically connected to a computer (e.g., a computer containing a set of executable instructions for performing methods described by the disclosure).

### EXAMPLES

### Example 1

### pLSC6 Score

### Patients

The pediatric AML leukemic stem cell (LSC) score was defined using data from 163 patients treated on the multicenter AML02 clinical trial with Affymetrix U133A microarray gene expression data obtained from diagnostic bone marrow specimens (Table 2). Patients with t[8;21], inv[16], or t[9;11] chromosome abnormalities were classified as low-risk AML. High-risk AML classification included presence of −7, FLT3-ITD mutation, t[6;9], acute megakaryoblastic leukemia (AMKL), treatment-related AML, or AML arising from MDS. Absence of low or high-risk group features was classified as standard-risk AML. Patients were randomized to receive high (3 g/m2, given every 12 h on days 1, 3 and 5) or low dose (100 mg/m2 given every 12 h on days 1-10) cytarabine along with daunorubicin and etoposide as a first course of chemotherapy with subsequent treatment tailored to response and risk classification. MRD positivity was defined as one or more leukemic cell per 1000 mononuclear bone-marrow cells (e.g., >0.1%). Event free survival (EFS) was defined as the time from study enrollment to induction failure, relapse, secondary malignancy, death, or study withdrawal for any reason, with event free patients censored on the date of last follow-up. Overall survival (OS) was defined as the time from study enrollment to death, with living patients censored on the date of last follow-up.

The validation cohort included 205 patients from Children's Oncology Group (COG) AAML0531 and AAML03P1 protocols with RNA seq and clinical outcome data available through the TARGET project database.

TABLE 2
Summary of Patient characteristics (AML02 cohort) that were included in this
example. Characteristics of patients that were part of the parent clinical
trial but were not included in the present analysis is also shown.

| | | | | Patients excluded | | |
|---|---|---|---|---|---|---|
| | | | | due to lack of | | |
| | | Patients included | | specimen | | |
| Characteristics | Level | in the study | | availability | | P value |
| Treatment Arm | HDAC | 74 | | 39 | | 0.225 |
| | LDAC | 87 | | 30 | | |
| | Not | 2 | | 0 | | |
| | Randomized | | | | | |
| Gender | Female | 74 | | 28 | | 0.595 |
| | Male | 89 | | 41 | | |
| Age Group | <10 | 83 | | 38 | | 0.663 |
| | >=10 | 80 | | 31 | | |
| Age (continuous) | | 8.79 | (0.0137~21.19) | 8.04 | (0.205~21.35) | 0.546 |
| Provisional Risk | High | 43 | | 33 | | 0.005 |
| | Low | 55 | | 14 | | |
| | Standard | 65 | | 22 | | |
| WBC Group | <30 | 91 | | 50 | | 0.026 |
| | >=30 | 72 | | 19 | | |
| WBC | | 55.32 | (0.013~513.6) | 35.64 | (0.017~409) | 0.019 |
| (continuous) | | | | | | |

| | | | | |
|---|---|---|---|---|
| Race | Black | 31 | 12 | 0.699 |
| | Other | 16 | 9 | |
| | Unknown | 1 | 1 | |
| | White | 115 | 47 | |
| Cytogenetic | 11q23 | 20 | 7 | 0.02 |
| Group | inv (16) | 21 | 5 | |
| | Miscell | 41 | 32 | |
| | Normal | 43 | 10 | |
| | t (8; 21) | 24 | 7 | |
| | t (9; 11) | 12 | 4 | |
| | Inevaluable | 2 | 4 | |
| MRDIND1 | Inevaluable | 8 | 6 | 0.578 |
| | Negative | 93 | 38 | |
| | Positive | 62 | 25 | |
| Event-free | 5 years | 0.604 ± 0.0386 | 0.548 ± 0.0602 | 0.3 |
| Survival | | | | |
| Overall Survival | 5 years | 0.707 ± 0.036 | 0.676 ± 0.0593 | 0.7 |

### Gene Expression Profiling

Gene expression profiling of leukemic blasts obtained at diagnosis in the AML02 discovery cohort was performed using GeneChip® Human Genome U133A [Affymetrix, Santa Clara, Calif.]. The MAS 5.0 algorithm was used to obtain normalized gene expression signals. All the gene expression data was loge transformed before analysis. For the validation cohort, publicly available RPKM (Reads per kilo base of transcript per million mapped reads data from TARGET database) were used. The study included 205 patients from AAML0531 and AAML03P1 clinical trials, with gene expression data available from diagnostic specimens (RNAseq data from specimen obtained at relapse were not included in this analysis). Log₂(RPKM+1) values were used for subsequent statistical analysis; TARGET dataset was observed to be enriched for patients with poor outcome.

### Pediatric LSC Score Signature

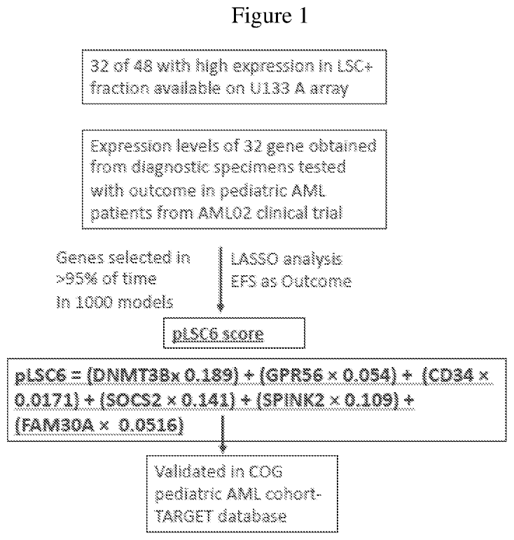

FIG. 1 illustrates one embodiments of an overall study design and implementation. Among 48 LSC-enriched genes previously identified, 32 were represented in the AML02 U133A microarray expression data set. A least absolute shrinkage and selection operator (LASSO) Cox regression model was fit, as implemented in glmnet package of R3.4.1 statistical software, to the expression of the 32 genes and the event-free survival (EFS) data of the AML02 study. To evaluate the variability and reproducibility of the LASSO Cox regression model estimates, the LASSO Cox regression fitting process was repeated for each of 1,000 bootstrap cohorts obtained by resampling subjects without replacement. Genes with non-zero coefficient estimates in at least 950 of these 1,000 bootstrap evaluations were retained. For each of these genes, the final model coefficient was the average of the coefficient estimates obtained for the set of bootstrap cohorts. A recursive partitioning survival model was also produced, as implemented in the rpart package, to dichotomize pLSC6 scores into “low” and “high” score groups to simplify reporting and graphing the association of pLSC6 with survival outcomes.

### Statistical Analysis

Survival analyses were performed using survival and survminer packages in R3.4.1. Event-free survival (EFS) and overall survival (OS) probabilities were estimated using the Kaplan-Meier method, and Cox proportional hazard models were used to compare the survival curves of patients within pLSC6 score groups as well as the association between each individual prognostic factor and survival outcomes. A Multivariable Cox proportional hazard model was used to evaluate the independent prognostic effect of the study covariates. Firth-penalized Cox regression was used to avoid monotone likelihoods and stabilize results for some analyses with small sample sizes. Wilcoxon rank-sum or Kruskal-Wallis tests were used for continuous variable comparisons between/among patient subgroups. Chi-square or fisher exact tests were used for testing association between categorical variables. A bootstrap procedure was used to compute a confidence interval for a ratio of hazard ratios (RHR) statistic comparing the strength of association of survival with LSC6 score model estimates, the LASSO Cox regression fitting process for each of 1,000 bootstrap cohorts obtained by resampling subjects without replacement. Genes with non-zero coefficient estimates in at least 950 of these 1,000 bootstrap evaluations were retained. For each of these genes, the final model coefficient was the average of the coefficient estimates obtained for the set of bootstrap cohorts. A recursive partitioning survival model, as implemented in the rpart package, was used to dichotomize pLSC6 scores into “low” and “high” score groups to simplify reporting and graphing the association of pLSC6 with survival outcomes.

### Expression of Six Leukemic Sternness Genes Defines a LSC6 Score of Prognostic Value

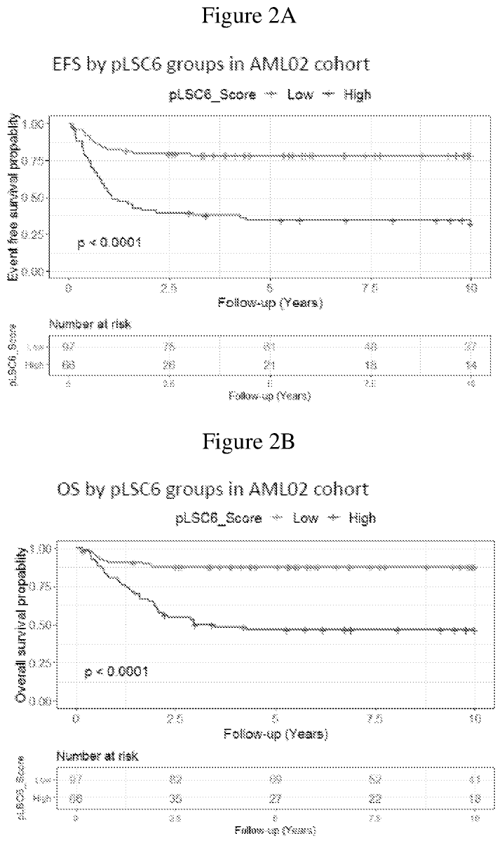

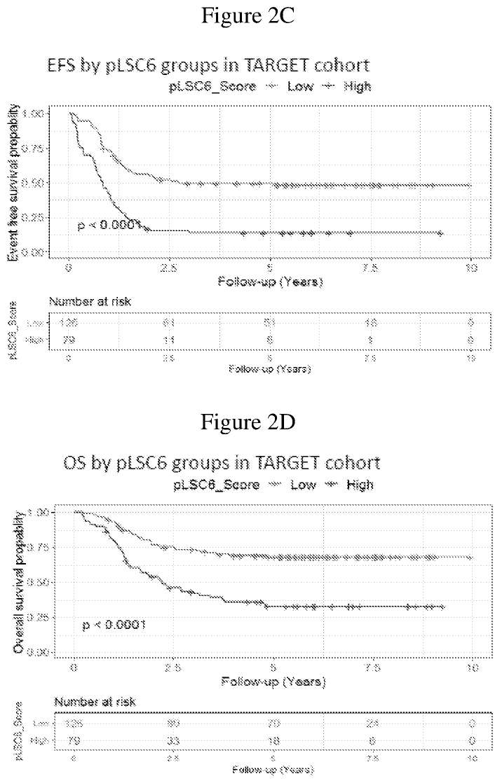

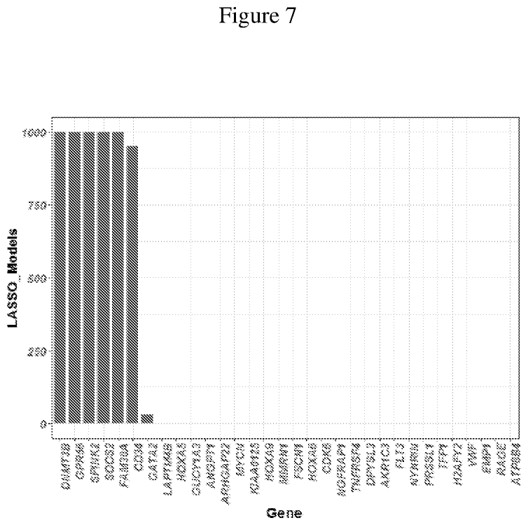

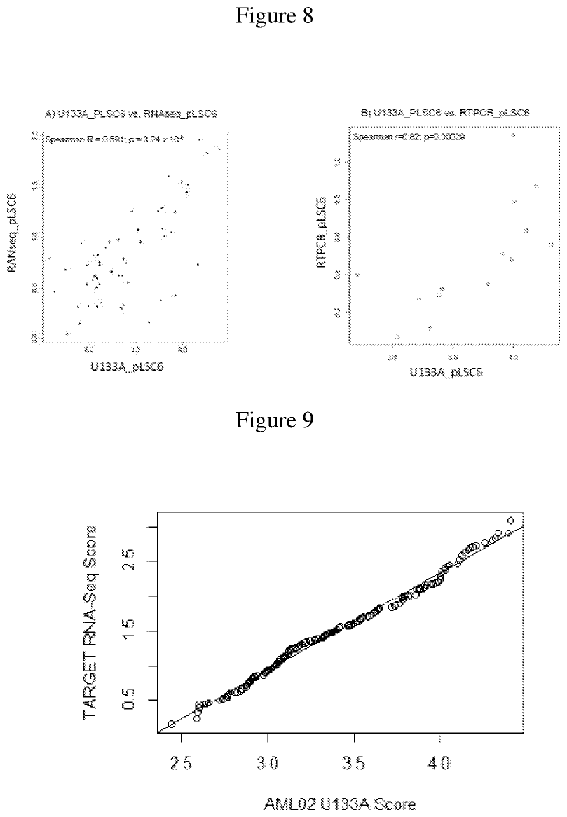

It was observed that 32 of 48 genes identified as over-expressed in LSCs were represented on the U133A microarray mRNA expression array. LASSO Cox regression model was used to model EFS with mRNA expression data (32 LSC genes) as predictors in 163 pediatric AML patients (model-development cohort) treated on AML02. Six genes were identified as important in at least 950 of 1,000 bootstrap replications of this analysis (FIG. 7). This rigorous model development process defined a six-gene pediatric LSC score (pLSC6) which was computed for each patient using gene expression weighted by the regression coefficients as defined in the equation pLSC6=(DNMT3B×0.189)+(GPR56×0.054)+(CD34×0.0171)+(SOCS2×0.141)+(SPINK2×0.109)+(FAM30A×0.0516). Each unit increase in pLSC6 was associated with a 4.34-fold increase in the rate of EFS events (p<0.00001, 95% CI=2.58-7.31) in a simple single-predictor Cox regression model. Recursive-partitioning Cox regression model was used to dichotomized pLSC6 with patients classified into low-pLSC6 score group (n=97 patients, 60%) or high-pLSC6 score group (n=66 patients; 40%). Comparison of patient characteristics between low pLSC6 and high-pLSC6 groups within AML02 is provided in Table 4, initial risk group assignment, cytogenetics and FLT3 status demonstrated significant difference by pLSC6 group classification. The five-year EFS of patients with low-pLSC6 score was 78.3 (95% CI=70.5-86.9), while that of patients with high-pLSC6 score was 34.5% (95% CI=24.7-48.2); HR=4.14 (95% CI=2.46-6.98; p<0.0001, FIG. 2A). Further, high-pLSC6 score was predictive of inferior OS in AML02 cohort (HR=5.18, 95% CI=2.67-10.1; p<0.0001, FIG. 2B). In subset of patients (n=55), RNAseq data was available, and the pLSC6 score was computed with the RNA-seq data using the coefficients defined above. The RNA-seq PLSC6 score strongly correlated with the U133A_pLSC6 score (Spearman R=0.591; p=3.24×10-6; FIG. 8). RT-PCR based quantification on subset of patients (n=14) within low and high pLSC6 score groups also demonstrated significant correlation between the pLSC6 derived using U133 or RT-PCR (Spearman R=0.82; p=0.00029).

Validation of pLSC6 as a Prognostic Score

To validate the prognostic value of pLSC6 in pediatric AML, the equation defined above was used to compute pLSC6 values in an independent cohort of 205 pediatric AML TARGET patients with clinical outcome and mRNA-seq expression data (model-validation cohort). It was observed that the distribution of pLSC6 values for the TARGET model-validation cohort had a very similar shape as that of the pLSC6 values for the AML02 model-discovery cohort (FIG. 9; QQ plot).

In a simple single-predictor cox model fit to the TARGET validation-cohort data, each unit increase in pLSC6 associated with a 1.91-fold increase in the rate of EFS failure events (p<0.0001; 95% CI=1.48-2.46). Recursive partitioning resulted in similar dichotomization of the

TARGET as observed in AML02 cohort with 60% of patients (n=126) patients within in low-pLSC6 group and 40% of patients (n=79) classified into high-pLSC6 group (patient characteristics by LSC6 group summarized in Table 4). In the TARGET cohort, the five year EFS of those with low-pLSC6 was 49.2 (95% CI=41.1-58.9), compared with 13.7 (95% CI=7.85-23.95) for those with high-pLSC6 (HR=2.86, 95% CI=2.02-4.04, p<0.0001, FIG. 2C).

Patients within high-pLSC6 also demonstrated inferior OS as compared to low-pLSC6 group within Target cohort (HR=2.81, 95% CI=1.85-4.28; p<0.0001, FIG. 2D). Table 3 provides a summary of univariate Cox regression results for association between study covariates with event free survival (EFS) and overall survival (OS) in AML02 the model-development and TARGET the model-validation cohorts.

pLSC6 is an Independent Prognostic Factor in the AML02 and TARGET Cohorts

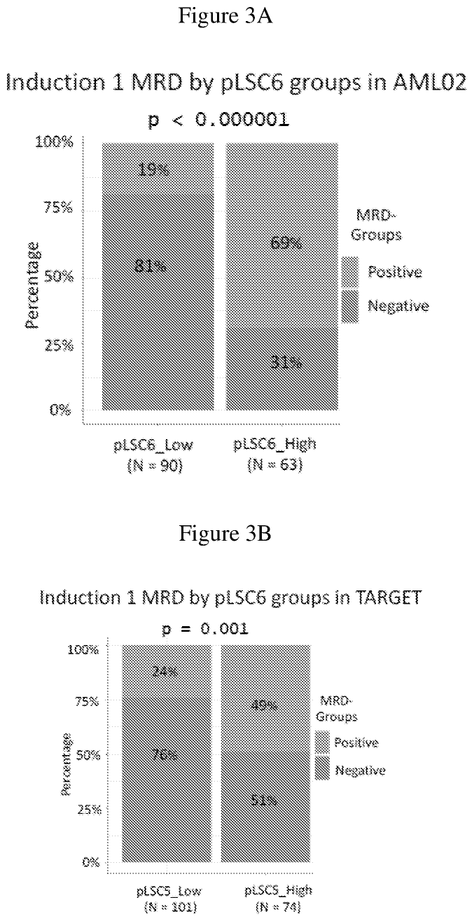

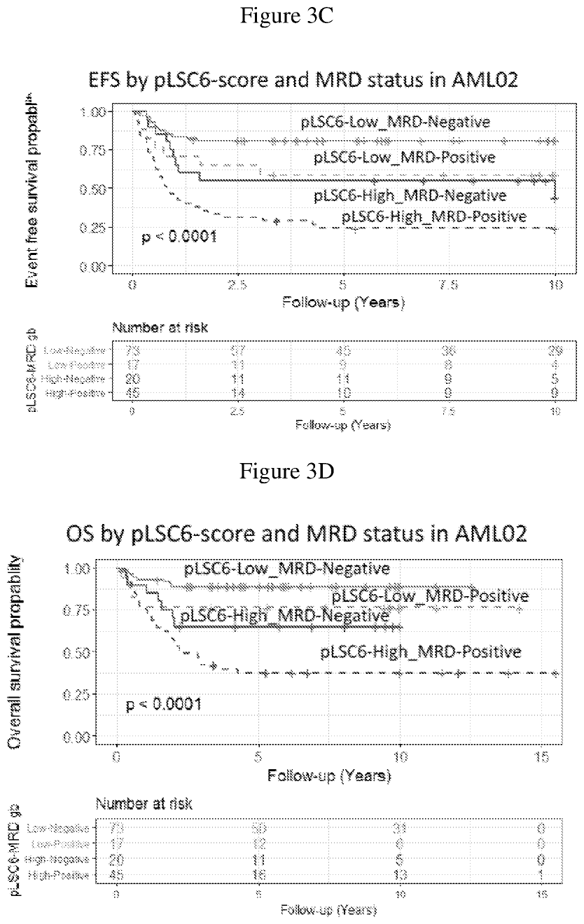

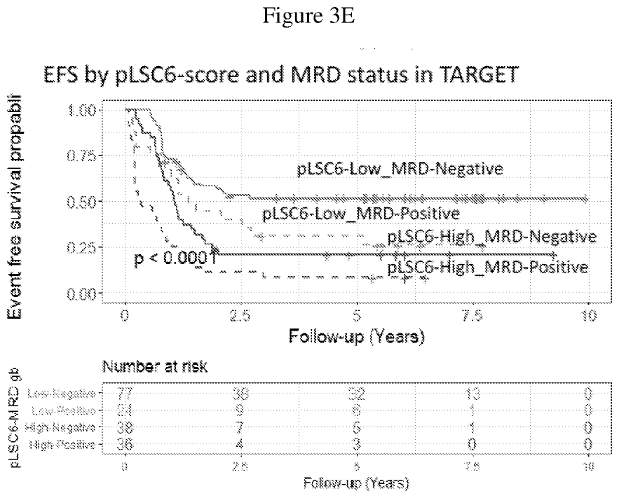

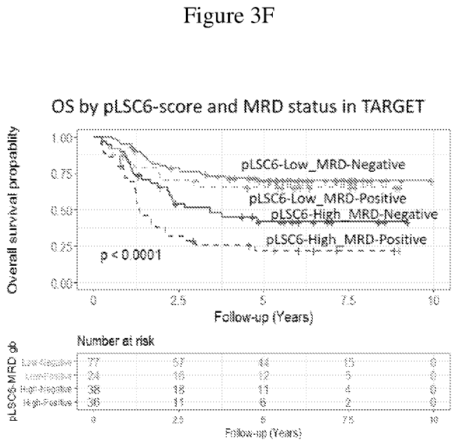

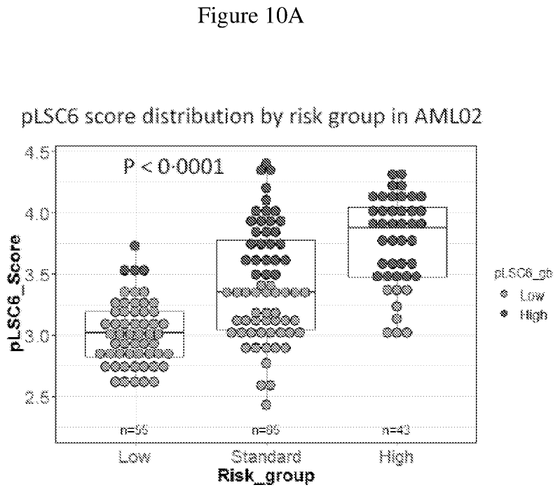

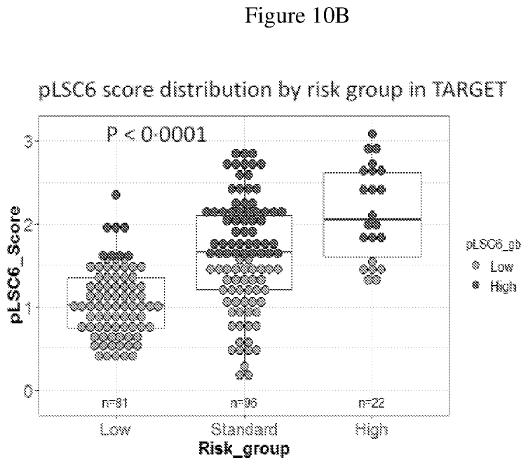

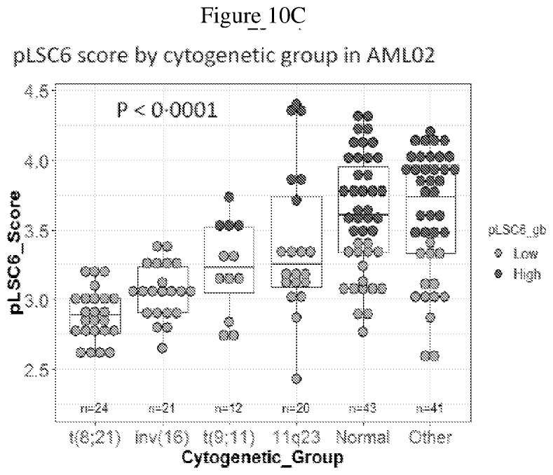

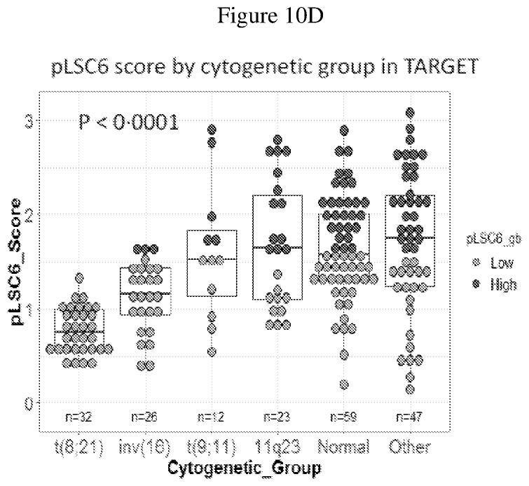

It was observed that pLSC6 provided prognostic information beyond that provided by minimal residual disease (MRD) and molecular risk classification in both the AML02 and TARGET cohorts. pLSC6 differed across molecular risk classification (p<0.0001 in both cohorts; FIGS. 10A-10D) and was strongly associated with MRD in both cohorts (AML02 cohort, p<0.0001, and Target cohort, p=0.001; FIG. 3A and 3B respectively).

pLSC6 provided additional prognostic information beyond that available from these two factors widely used in clinical practice. In the AML02 cohort, the five-year EFS was 80.8 ±4.6% in MRD- patients with low-pLSC6 score; 58.8 ±11.9% in MRD+patients with low-pLSC6 score, 55 ±11.1% in MRD-patients with high-pLSC6 score, and 24.1 ±6.4% in MRD+ patients with high-pLSC6 scores. A Cox model with dichotomized pLSC6 score and MRD as predictors found that high-pLSC6 score is associated with a 2.67-fold increased rate of EFS failure (95% CI=1.48-4.81, p=0.001) and 3.31-fold increased rate of death (95% CI=1.58-6.97, p=0.0015) relative to low-pLSC6 score in the AML02 cohort holding MRD constant. A similar model found high-pLSC6 score associated with a 2.38-fold increase in EFS failure rate (95% CI=1.64-3.46, p<0.0001) and 2.72-fold increase in death rate (95% CI=1.71-4.36, p<0.0001) in the TARGET cohort. FIGS. 3C-3F show EFS and OS in both AML02 (FIG. 3C and 3D) and Target cohorts (FIG. 3E and 3F) by pLSC6 and MRD status. These results indicate that pLSC6 provides additional prognostic information not captured by MRD.

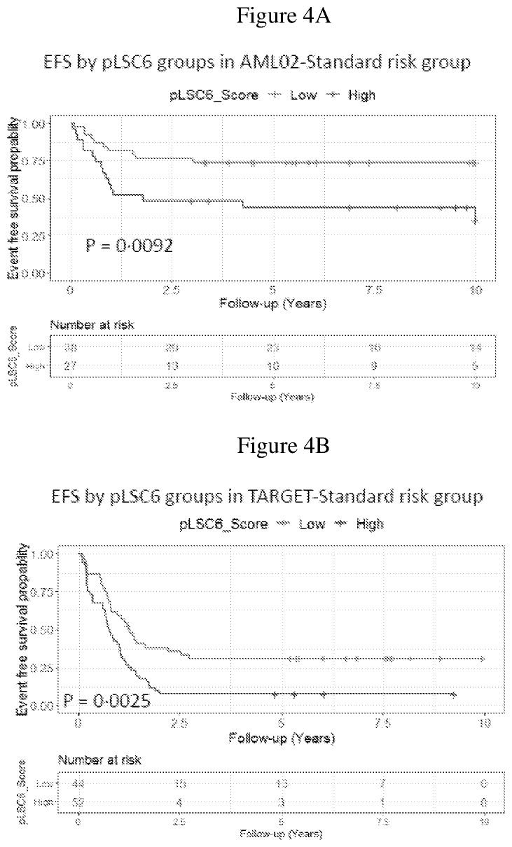

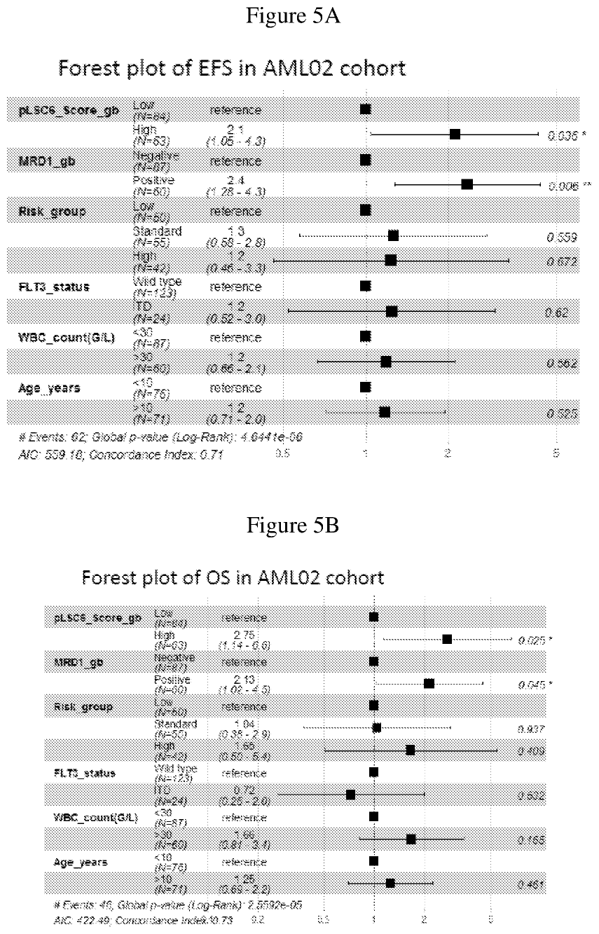

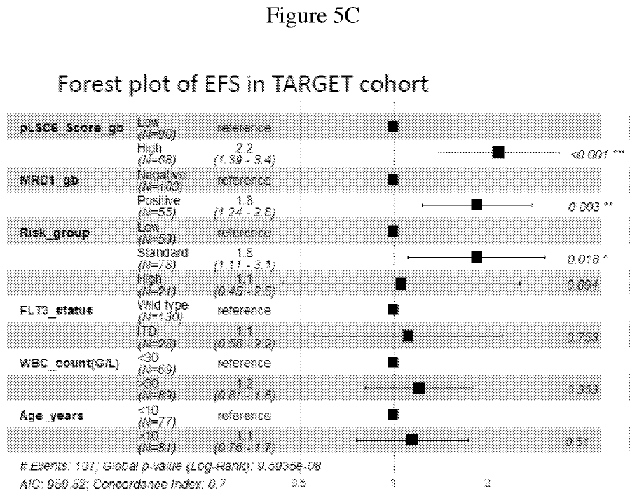

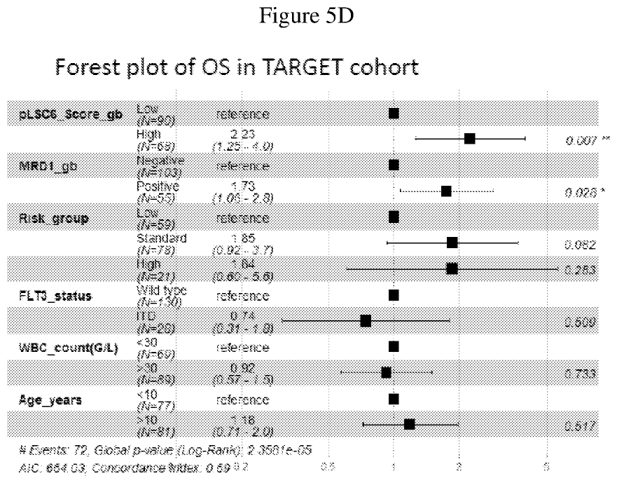

pLSC6 also provides prognostic information not captured by molecular risk classification. Within each risk group in both cohorts, it was observed that high-pLSC6 score patient had worse prognosis than low-pLSC6 score patients. In the AML02 cohort, Cox models with dichotomized pLSC6 score and molecular risk group (low, standard and high) found that high-pLSC6 score associated with worse EFS (HR=3.45; 95 CI=1.83-4.51; p=0.0001) and OS (HR=3.93; 95%CI=1.78-8.72; p=0.0007). In the TARGET cohort, similar results were obtained for EFS (HR=2.16; CI=1.47-3.17; p<0.0001) and OS (HR=2.03; CI=1.26-3.26; p=0.004). pLSC6 was also observed to be significantly associated with EFS in standard risk patients of the AML02 cohort (HR=2.86; 95% CI=1.29-6.33, p=0.009) and of the TARGET cohort (HR=2.04; 95% CI=1.28-3.24, p=0.002), as shown in FIG. 4. pLSC6 also better risk-stratifies standard risk patients. Even after adjusting for risk group, MRD, FLT3 status, diagnostic WBC count, and age, dichotomized pLSC6 remained an independent predictor of worse EFS and OS in both cohorts (FIG. 5).

pLSC6 was also significantly associated with outcome within four of the five major treatment arms represented in the AML02 and TARGET data sets. Single-predictor Cox regression models indicate that each unit increase of the pLSC6 score associated with worse EFS in the low-dose ara-C arm of AML02 (HR=4.15, 95% CI: 2.02, 8.52; p=0.0001), the high-dose ara-C arm of AML02 (HR=4.54; 95% CI: 2.05, 10.06; p=0.0002), the AAMLO3P1 protocol (HR=2.1; 95% CI: 1.10, 4.02; p=0.025), and the standard arm of AAML0531 in the TARGET cohort (HR=2.02; 95% CI: 1.31, 3.14;p p=0.0016). In the GO arm of AAML0531, each unit increase in pLSC6 showed an association with worse EFS (HR=1.36; 95% CI: 0.86, 2.14; p=0.18). Similar results were obtained for OS. Each unit increase in pLSC6 score was significantly associated with worse OS in the low-dose ara-C arm of AML02 (HR=4.3; 95% CI=1.87, 10.04; p=0.0006), the high-dose ara-C arm of AML02 (HR=7.07, 95% CI: 2.55-19.62; p=0.0002), and the standard arm of the AAML0531 protocol (HR=1.76, 95% CI: 1.03, 2.98; p=0.037). Each unit increase in pLSC6 had an association with worse OS in the GO arm of AAML0531 (HR=1.64, 95% CI: 0.96, 2.79; p=0.069) and the AAMLO3P1 protocol (HR=2.3, 95% CI: 0.99, 5.38; p=0.053).

pLSC6 to Identify Candidates for Transplant

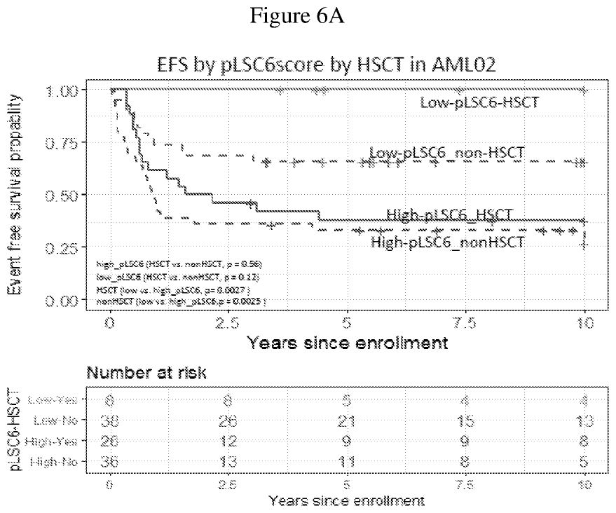

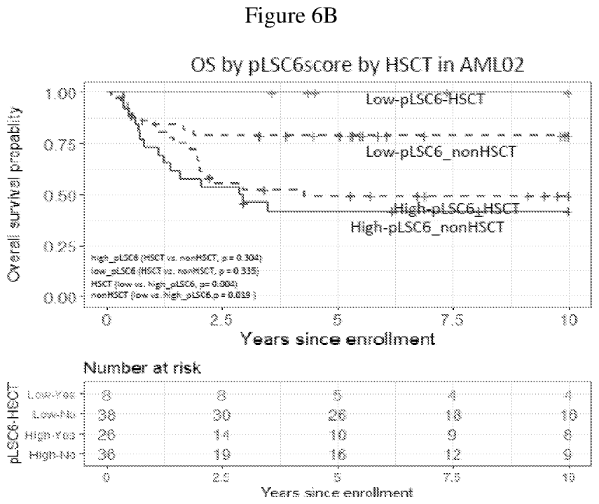

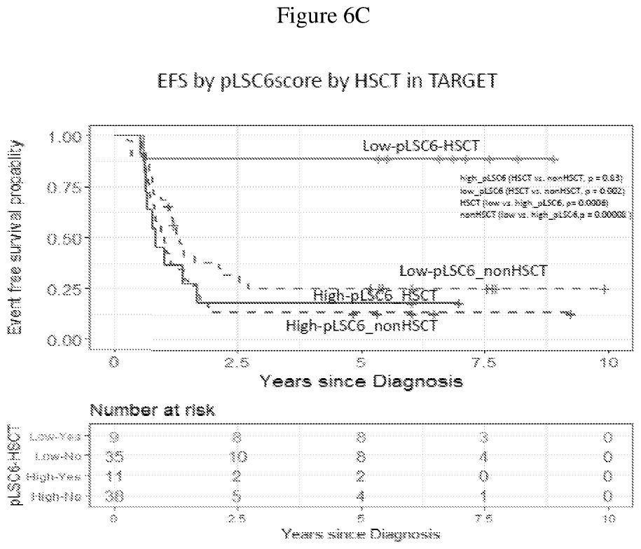

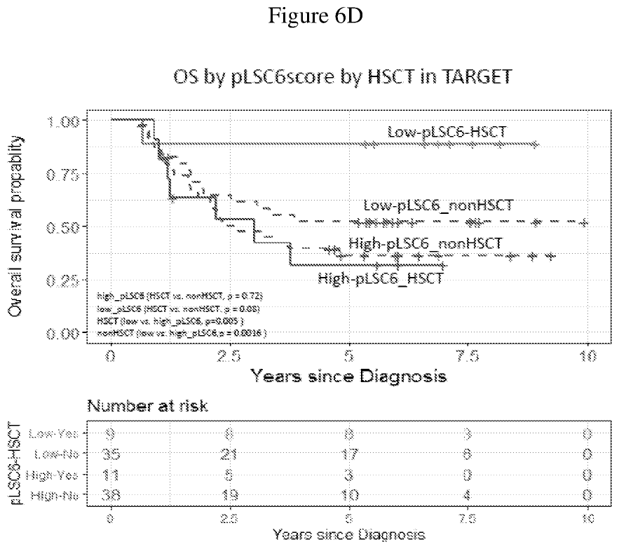

Among standard and high risk patients of both the AML02 and TARGET cohorts, transplant was associated with better outcomes compared to chemotherapy alone for patients with low-pLSC6 score, but transplant and chemotherapy alone showed similarly dismal outcomes for patients with high-pLSC6 score (FIGS. 6A-6D). Among low-pLSC6 score patients, transplant was associated with a statistically suggestive and clinically substantial improvement in EFS in the AML02 cohort (HR=0.18; 95% CI: 0.001, 1.40; p=0.12) and an improvement that was both clinically substantial and statistically significant in the TARGET cohort (HR=0.14; 95% CI: 0.015,0.54; p=0.002). Also, among low PLSC6 score patients, those with transplants had notably better OS in the AML02 cohort (HR=0.30; 95% CI: 0.002, 2.52; p=0.34) and significantly better OS in the TARGET cohort (HR=0.28; 95% CI: 0.03, 1.15; p=0.08).

In contrast, data described in this example indicates that transplant may not provide a clinical benefit for patients with high-pLSC6 scores. Among AML02 patients with high-pLSC6 scores, Cox regression modeling found that transplant had an association with slightly worse EFS (HR=1.22, 95% CI: 0.6, 2.44; p=0.56) and OS (HR=1.43, 95% CI: 0.71, 2.85; p=0.30; Supplementary Note 1). Likewise, among TARGET patients with high-LSC6 scores, Cox regression modeling found that transplant had an association with slightly worse EFS (HR=1.08, 95% CI: 0.50, 2.14; p=0.83) and OS (HR=1.16; 95% CI: 0.48, 2.52; p=0.72). These data indicate that pLSC6 may identify patients who are most likely to benefit from transplant.

Comparison of pLSC6 with LSC17

A quantitative comparison between pLSC6 and LSC17, a previously developed adult AML scoring system, was performed to assess the models as predictors of EFS and OS in the pediatric AML TARGET cohort by computing 95% bootstrap confidence intervals (95% BCI) for the ratio of hazard ratios (RHR) expressed as the hazard ratio for the interquartile range of pLSC6 relative to the hazard ratio for the interquartile range of LSC17.

RHR=1 indicates that pLSC6 and LSC17 have the same strength of association with the survival outcome; RHR>1 indicates that pLSC6 has a stronger association with survival than LSC17; and RHR<1 indicates that pLSC6 has a weaker association with survival than LSC17. In the TARGET cohort, the association of pLSC6 with EFS was 1.21 times stronger than the association of pLSC17 with EFS (RHR=1.21; 95% BCI=0.95, 1.57). pLSC6 and LSC17 had a similar strength of association with OS (RHR=1.18; 95% BCI=0.90, 1.56).

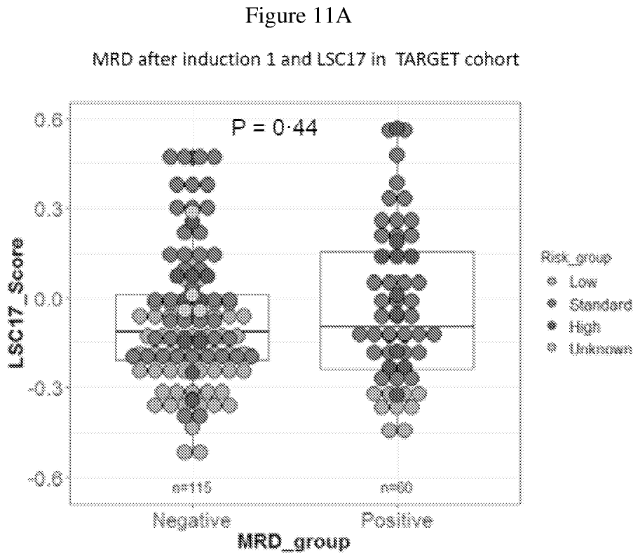

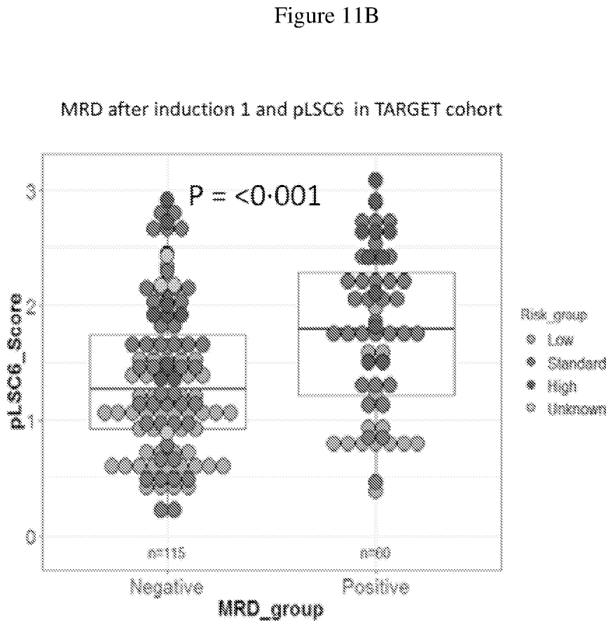

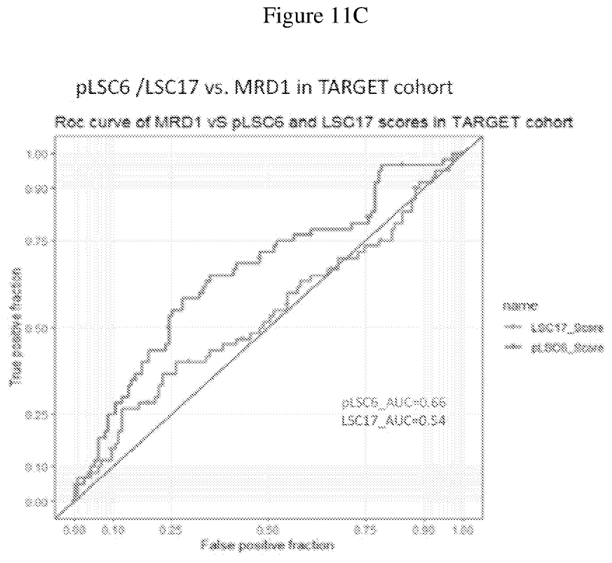

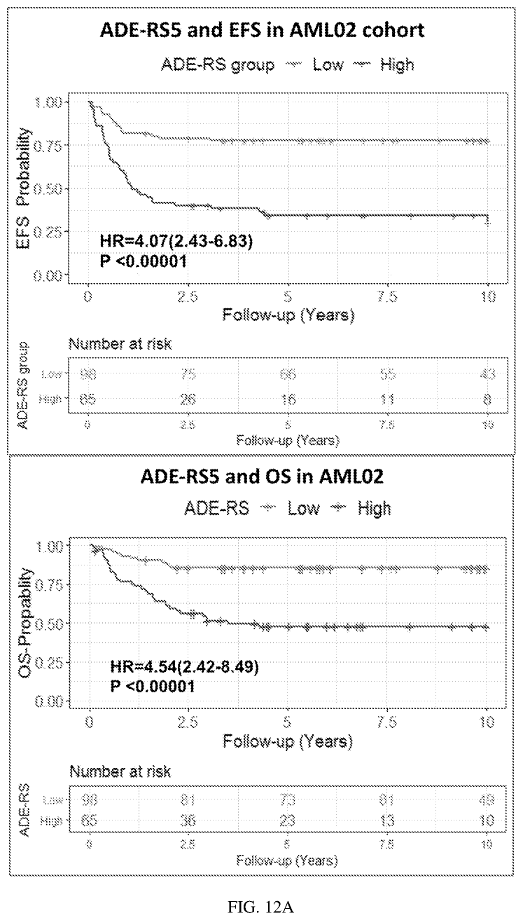

Additionally, within the TARGET cohort, it was observed that while LSC17 was not significantly associated with induction 1 MRD (p=0.44), pLSC6 was significantly associated with induction 1 MRD (p<0.0001), as shown in FIGS. 11A-11C, indicating that pLSC6 is a better predictor of EFS for pediatric AML than LSC17.

TABLE 3
Univariate Cox regression results for association between study covariates with event free survival (EFS) and overall survival (OS) in
AML02 and TARGET cohorts.

| | Event free survival (EFS) time | | | | | | Overall survival (OS) time | | | | | |
|---|---|---|---|---|---|---|---|---|---|---|---|---|
| | AML02 | | | TARGET | | | AML02 | | | TARGET | | |
| | Hazard ratio | | | Hazard ratio | | | Hazard ratio | | | Hazard ratio | | |
| | (95% CI) | | P-value | (95% CI) | | P-value | (95% CI) | | P-value | (95% CI) | | P-value |
| Low-pLSC6 | — | | — | — | | — | — | | — | — | | — |
| High-pLSC6 | 4.14 | (2.46-6.98) | <0.0001 | 2.86 | (2.02-4.04) | <0.0001 | 5.18 | (2.67-10.1) | <0.0001 | 2.81 | (1.85-4.28) | <0.0001 |
| Low risk | — | | — | — | | — | — | | — | — | | — |
| group | | | | | | | | | | | | |
| Standard Risk | 1.99 | (0.965-4.12) | 0.062 | 2.95 | (1.98-4.39) | <0.0001 | 2.27 | (0.873-5.91) | 0.093 | 3.01 | (1.81-5.03) | <0.0001 |
| group | | | | | | | | | | | | |
| High risk | 4.56 | (2.29-9.05 | <0.0001 | 2.26 | (1.24-4.14) | 0.007 | 6.18 | (2.55-14.99) | <0.0001 | 2.75 | (1.31-5.76) | 0.007 |
| group | | | | | | | | | | | | |
| Cytogenet- | — | | — | — | | — | — | | — | — | | — |
| ically | | | | | | | | | | | | |
| normal | | | | | | | | | | | | |
| Inv(16) | 0.143 | (0.033-0.611) | 0.009 | 0.926 | (0.509-1.68) | 0.8 | 0.0932 | (0.012-0.701) | 0.021 | 0.641 | (0.267-1.48) | 0.298 |
| t (8; 21) | 0.368 | (0.139-0.977) | 0.0045 | 0.683 | (0.376-1.24) | 0.211 | 0.293 | (0.085-0.998) | 0.045 | 0.763 | (0.366-1.58) | 0.469 |
| t (9; 11) | 0.731 | (0.276-1.94) | 0.529 | 0.745 | (0.315-1.76) | 0.503 | 0.559 | (0.164-1.91) | 0.353 | 0.818 | (0.284-2.35) | 0.711 |
| 11q23 | 0.835 | (0.382-1.82) | 0.65 | 2.09 | (1.22-3.57) | 0.006 | 0.828 | (0.343-1.99) | 0.674 | 1.91 | (1.008-3.63) | 0.047 |
| Other | 1.04 | (0.573-1.89) | 0.894 | 1.82 | (1.15-2.86) | 0.009 | 0.827 | (0.4413-1.66) | 0.592 | 1.96 | (1.15-3.35) | 0.013 |
| aberrations | | | | | | | | | | | | |
| FLT3-WT | — | | — | — | | — | — | | — | — | | — |
| Mutation | 1.05 | (0.326-3.38) | 0.934 | 0.91 | (0.47-1.73) | 0.76 | 0.493 | (0.067-3.61) | 0.486 | 0.56 | (0.23-1.39) | 0.21 |
| FLT3-ITD | 2.69 | (1.54-4.71) | 0.0005 | 1.45 | (0.92-2.29) | 0.11 | 2.74 | (1.46-5.14) | 0.002 | 1.36 | (0.79-2.34) | 0.27 |
| WBC >30(G/L) | 2.69 | (1.54-4.71) | 0.5 | 0.95 | (0.67-1.34) | 0.78 | 1.36 | (0.77-2.42) | 0.287 | 0.76 | (0.51-1.15) | 0.19 |
| BM | 1.36 | (0.813-2.26) | 0.244 | 1.07 | (0.76-1.5) | 0.71 | 1.55 | (0.852-2.83) | 0.15 | 0.98 | (0.65-1.48) | 0.94 |
| Blast >70% | | | | | | | | | | | | |
| Peripheral | 1.45 | (0.889-2.36) | 0.136 | 0.84 | (0.59-1.18) | 0.32 | 1.94 | (1.08-3.48) | 0.026 | 0.79 | (0.53-1.21) | 0.29 |
| Blast >50% | | | | | | | | | | | | |
| Age >10 years | 1.15 | (0.707-1.87) | 0.572 | 0.84 | (0.59-1.18) | 0.61 | 1.18 | (0.669-2.09) | 0.561 | 1.23 | (0.82-1.86) | 0.32 |

TABLE 4
Characteristics of 163 patients enrolled in AML02, the model development
cohort and 205 patients from TARGET dataset (expression data from only
diagnostic specimens were utilized) the model-validation cohort.

| | | AML02 | | | | | |
|---|---|---|---|---|---|---|---|
| | | pLSC6- | pLSC6- | | TARGET | | |
| | | Low | High | P-value | pLSC6_Low | pLSC6_High | P-value |
| Risk groups | Low | 51 | 4 | <0.0001 | 73 | 8 | <0.0001 |
| | Standard | 38 | 27 | | 44 | 52 | |
| | High | 8 | 35 | | 6 | 16 | |
| | Unknown | | | | 3 | 3 | |
| Cytogenetic | Normal | 16 | 27 | <0.0001 | 31 | 28 | <0.0001 |
| groups | Inv(16) | 21 | 0 | | 23 | 3 | |
| | t(8; 21) | 24 | 0 | | 32 | 0 | |
| | t(9; 11) | 8 | 4 | | 7 | 5 | |
| | 11q23 | 14 | 6 | | 10 | 13 | |
| | Other | 13 | 28 | | 20 | 27 | |
| | Unknown | 1 | 1 | | 3 | 3 | |
| FLT3_Status | Wild type | 87 | 42 | <0.0001 | 101 | 56 | 0.002 |
| | Mutation | 5 | 3 | | 14 | 3 | |
| | ITD | 4 | 21 | | 11 | 20 | |
| WBC count | <30 G/L | 52 | 39 | 0.595 | 46 | 36 | 0.253 |
| | >30 G/L | 45 | 27 | | 80 | 43 | |
| Bone marrow | <70% | 52 | 34 | 0.726 | 59 | 28 | 0.144 |
| blast % | >70% | 36 | 28 | | 67 | 51 | |
| Peripheral | <50% | 54 | 31 | 0.23 | 44 | 31 | 0.854 |
| blast % | >50% | 38 | 34 | | 82 | 48 | |
| Age | <10 years | 47 | 36 | 0.546 | 25 | 18 | 0.634 |
| | >10 years | 50 | 30 | | 26 | 33 | |
| AML02 | LDAC | 49 | 38 | 0.589 | — | — | NA |
| treatment | HDAC | 47 | 27 | | — | — | |
| arm | Not | 1 | 1 | | — | — | |
| | randomized | | | | | | |
| TARGET_COG | AAML0531 | — | — | NA | 92 | 59 | 0.919 |
| Protocol | AAML03P1 | — | — | | 34 | 20 | |
| Race | Black | 18 | 13 | 0.961 | 12 | 8 | 0.689 |
| | White | 69 | 46 | | 97 | 57 | |
| | Other | 9 | 7 | | 12 | 7 | |
| | Unknown | 1 | | | 5 | 7 | |
| Gender | Female | 45 | 28 | 0.882 | 53 | 36 | 0.727 |

### Example 2

### ADE-RS5 Score

Cytarabine, daunorubicin and etoposide (ADE) are commonly used for remission and intensification of pediatric acute myeloid leukemia (AML). However, development of drug resistance is a major cause of treatment failure. In this example, a comprehensive evaluation of expression levels of genes of pharmacological significance (pharmacokinetic/pharmacodynamic) to ADE was performed and a drug response score predictive of treatment outcomes in pediatric AML patients was derived.

This study included 163 cases (median age=8.79 year, range=(0.013-21.1)) with AML enrolled in the multicenter AML02 clinical trial (ClinicalTrials.gov Identifier: NCT00136084) with Affymetrix U133A microarray gene expression and clinical data available. A penalized LASSO regression algorithm (glmnet R-package) was used to fit a cox regression model on diagnostic leukemic cell gene expression levels of 66 genes of pharmacological significance to ADE and Event Free survival (EFS) as outcome. To evaluate the variability and reproducibility of the LASSO Cox regression model estimates, the LASSO Cox regression fitting process was repeated for each of 1,000 leave-10%-out cross-validation evaluations. Thus after running one thousand (1000) bootstraps of LASSO regression models with event free survival (EFS) as the outcome variable, five genes that were represented in at least 95% of the models were selected to build an ADE-Response Score (ADE-RS) equation. For each of these genes, the final model coefficient was the average of the coefficient estimates obtained for the set of cross-validation evaluations. Patients were classified into low or high score groups using recursive portioning implemented in Rpart-Rpackage and evaluated for association with minimal residual disease after induction I (MRD1), EFS and overall survival(OS). ADE response score equation was further validated using RNA-Seq gene-expression data obtained from diagnostic samples of 603 pediatric AML patients enrolled in Children's Oncology Group (COG) AAML0531 and AAML03P1 treatment protocols.

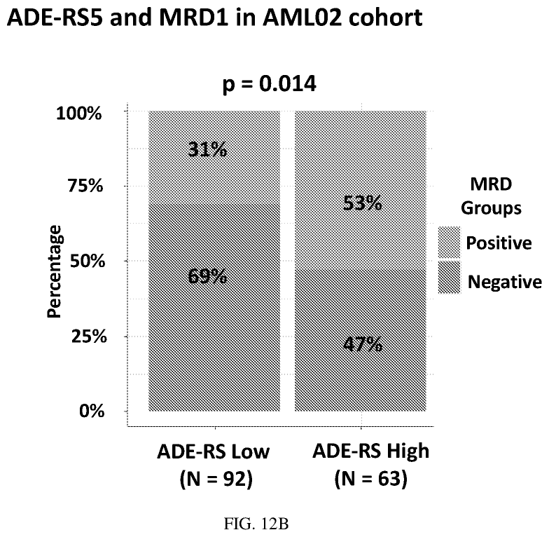

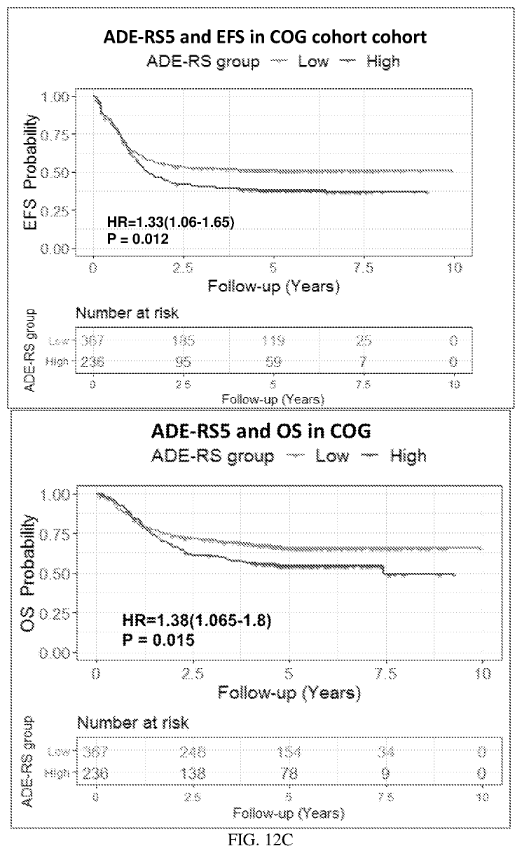

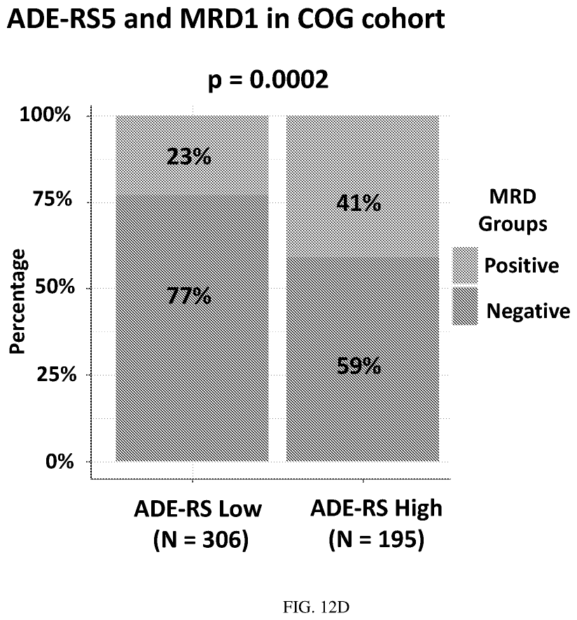

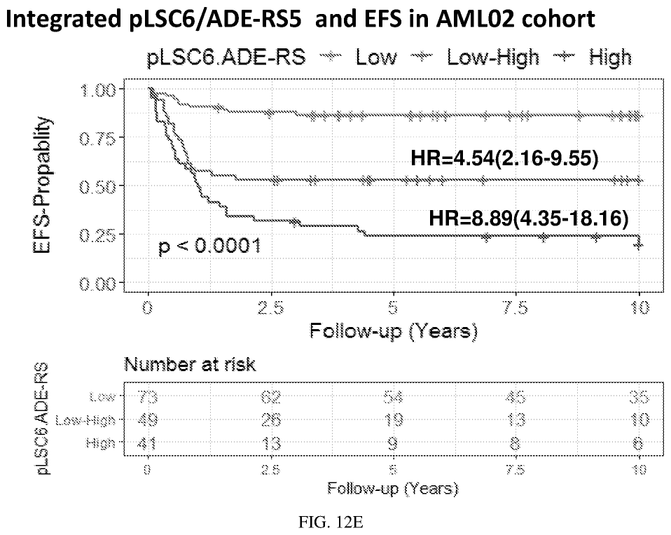

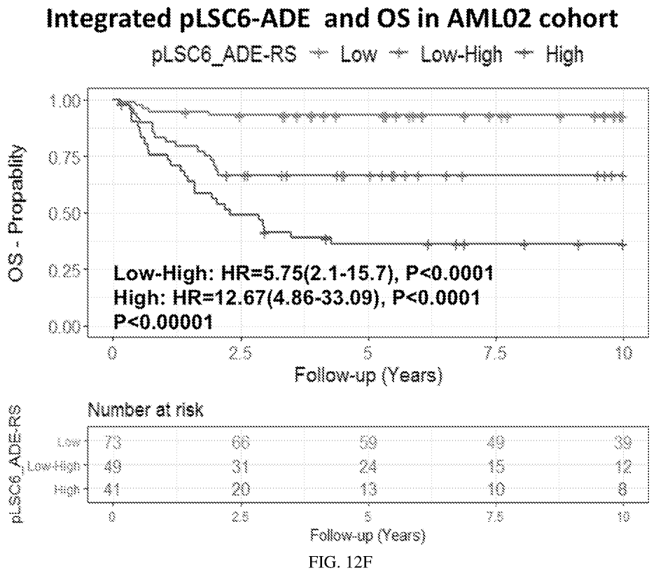

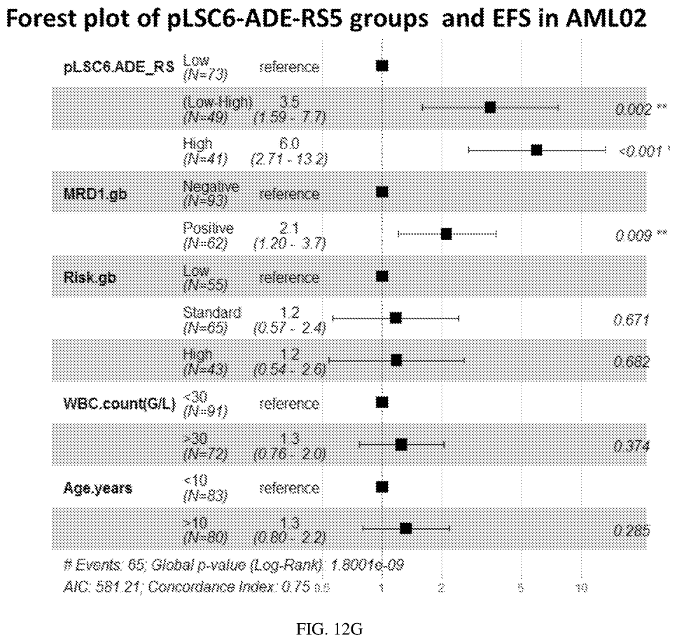

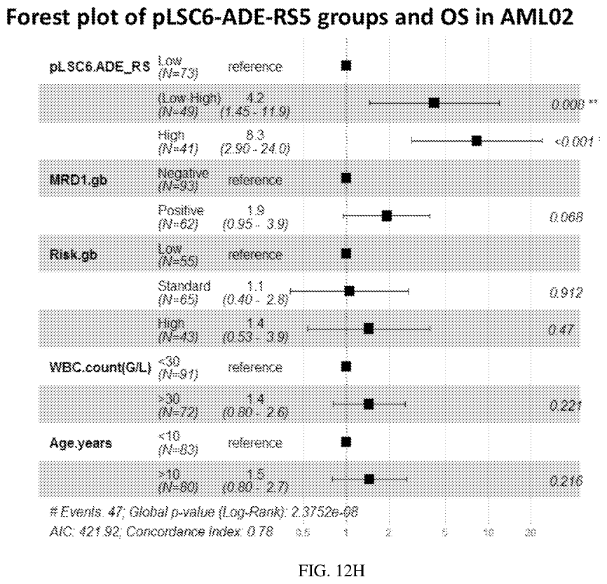

After applying LASSO regression, the following algorithm was defined: ADE-RS=(0.128×DCTD)−(0.0993×TOP2A)+(0.212×ABCC1)−(0.113×MPO)−(0.126×CBR1) to develop ADE-response score of 5 genes (ADE-RS5), followed by classifying patients into low (60%; 98 patients) or high (40%; 65 patients) score groups. Patients in the high ADE-RS5 group had significantly worse EFS (HR=4.07(2.43-6.84), P<0.0001; FIG. 12A) and OS (HR=4.54(2.42-8.49), P<0.0001, FIG. 12A) and higher proportion of MRD1 positive patients (P=0.014; FIG. 12B) compared to patients in the low ADE-RS5 group. Results were validated in an independent COG cohort, where patients in the high score group demonstrated higher MRD1 positivity (P=0.0005; FIG. 12D), inferior EFS (HR=1.32(1.01-1.73), P=0.044; and inferior OS (HR=1.38(1.065-1.8); FIG. 12C).

The six-gene leukemic stem cell score (pLSC6 score) described in Example 1 was integrated with ADE-RS5. Significantly better prediction of treatment outcomes in AML02, COG and TCGA cohorts were observed. Based on pLSC6 and ADE-response scores, patients were classified into three groups; 1) Low/Low:pLSC6/ADE-RS5; for patients with low pLSC6 and low ADE-RS; 2) Low/High:pLSC6/ADE-RS5: for patients in low pLSC6 and high ADE-RS5 or vice versa; and 3) High/High:pLSC6/ADE-RS5: for patients in high pLSC6 and high ADE-RS. In all study cohorts, patients in low/low pLSC6-ADE-RS5 group demonstrated better outcomes compared to the low-high and the high/high score groups (EFS in AML02 cohort; FIG. 12E and OS; FIG. 12F).

In a multivariable cox-regression models that included pLSC6-ADE response score groups, MRD1 status, risk groups, WBC at diagnosis, and age in AML02 cohort, high pLSC6-ADE score group was found significantly associated with poor EFS (HR=6.0(2.71-13.2), P<0.00001; FIG. 12G) and was a significant predictor of poor OS (HR=8.3(2.9-24), P<0.00001; FIG. 12H).

In summary, a pharmacological response score focused on key genes of PK/PD significance to ADE was defined. The response score was further integrated with pLSC6 score to improve treatment outcome prediction in AML patients across different clinical trials. ADE-RS was composed of five genes: DCTD, which is a deaminase, involved in ara-C inactivation; CBR1, a carbonyl reductase involved in inactivation of daunorubicin (DNR); MPO, myeloperoxidase, an etoposide activator; ABCC1, an efflux transporter of DNR and etoposide; and TOP2A, DNA topoisomerase II alpha, which is a target for DNR and etoposide.

1. A method for obtaining a pLSC6 score result in a subject having leukemia, the method comprising:
measuring a level of an RNA transcript of each of a set of genes consisting of DNMT3B, GPR56, CD34, SOCS2, SPINK2, and FAM30A in a biological sample obtained from a subject having leukemia; normalizing the levels against a level of at least one reference RNA transcript in the biological sample to provide normalized levels each of the RNA transcripts; weighting each of the normalized levels of the set to produce a weighted set; calculating a pLSC6 score for the subject using the weighted set; and creating a report comprising the pLSC6 score.

2. The method of claim 1, wherein the leukemia is acute myeloid leukemia (AML).

3. The method of claim 1, wherein the AML is pediatric AML or wherein the subject is less than 19 years of age.

4. The method of claim 1, wherein the biological sample is a blood sample, spinal fluid sample, or tissue sample.

5. The method of claim 4, wherein the tissue sample comprises bone marrow cells and/or leukemic blast cells.

6. The method of claim 1, wherein the RNA transcript is an mRNA transcript.

7. The method of claim 1, wherein the measuring comprises determining the RNA transcript level of each of the set of genes by a hybridization-based assay.

8. The method of claim 7, wherein the hybridization-based assay comprises a microarray assay or quantitative RT-PCR.

9. The method of claim 1, wherein the measuring comprises determining the RNA transcript level of each of the set of genes by a nucleic acid sequencing assay.

10. The method of claim 9, wherein the nucleic acid sequencing assay comprises nanopore sequencing, next-generation sequencing, high-throughput sequencing, or digital gene expression.

11. The method of claim 1, wherein the weighting comprises fitting a COX-LASSO regression model to the normalized levels of the set.

12. The method of claim 1, wherein the weighted set comprises at least one of the following regression coefficient values: (DNMT3B×0.189), (GPR56 ×0.054), (CD34×0.0171), (SOCS2×0.141), (SPINK2×0.109), or (FAM30A×0.0516).

13. The method of claim 1, wherein the pLSC6 score is calculated using the following algorithm:
pLSC6=(DNMT3B×0.189)+(GPR56 ×0.054)+(CD34×0.0171)+(SOCS2×0.141)+(SPINK2×0.109)+(FAM30A×0.0516).

14. The method of claim 1, wherein the report designates the subject as a “low-pLSC6” or a “high-pLSC6” subject.

15. The method of claim 1, wherein the report designates the subject as a candidate for transplant therapy, optionally wherein the transplant therapy comprises hematopoietic stem cell transplantation (HSCT).

16-35. (canceled)

36. A method for obtaining an ADE-RS5 score result in a subject having leukemia, the method comprising:
measuring a level of an RNA transcript of each of a set of genes consisting of DCTD, CBR1, MPO, ABCC1, and TOP2A in a biological sample obtained from a subject having leukemia; normalizing the levels against a level of at least one reference RNA transcript in the biological sample to provide normalized levels each of the RNA transcripts; weighting each of the normalized levels of the set to produce a weighted set; calculating an ADE-RS5 score for the subject using the weighted set; and creating a report comprising the ADE-RS5 score.

37. The method of claim 36, wherein the leukemia is acute myeloid leukemia (AML).

38. The method of claim 36, wherein the AML is pediatric AML or wherein the subject is less than 19 years of age.

39. The method of claim 36, wherein the biological sample is a blood sample, spinal fluid sample, or tissue sample.

40-70. (canceled)

71. A method for obtaining a pLSC6/ADE-RS5 score result in a subject having leukemia, the method comprising:
(i) measuring a level of an RNA transcript of each of a set of genes consisting of DNMT3B, GPR56, CD34, SOCS2, SPINK2, and FAM30A in a biological sample obtained from a subject having leukemia; normalizing the levels against a level of at least one reference RNA transcript in the biological sample to provide normalized levels each of the RNA transcripts;
weighting each of the normalized levels of the set to produce a first weighted set; calculating a pLSC6 score for the subject using the first weighted set;
(ii) measuring a level of an RNA transcript of each of a set of genes consisting of DCTD, CBR1, MPO, ABCC1, and TOP2A in a biological sample obtained from a subject having leukemia; normalizing the levels against a level of at least one reference RNA transcript in the biological sample to provide normalized levels each of the RNA transcripts; weighting each of the normalized levels of the set to produce a second weighted set; calculating an ADE-RS5 score for the subject using the second weighted set; and
(iii) creating a report comprising the pLSC6/ADE-RS5 score.

72-92. (canceled)
